(19) United States
(12) Patent Application Publication
(10) Pub. No.: US 2016/0137921 A1
(43) Pub. Date: May 19, 2016

(54) POLYMERIZABLE COMPOUND, AND LIQUID CRYSTAL COMPOSITION PRODUCED USING SAME

(71) Applicant: DIC CORPORATION, Tokyo (JP)

(72) Inventors: Masanao Hayashi, Kita-adachi-gun (JP); Masahiro Horiguchi, Kita-adachi-gun (JP); Tetsuo Kusumoto, Kita-adachi-gun (JP)

(73) Assignee: DIC CORPORATION, Tokyo (JP)

(21) Appl. No.: 14/778,795

(22) PCT Filed: Mar. 18, 2014
(86) PCT No.: PCT/JP2014/057278

§ 371 Date: Jan. 8, 2016

(30) Foreign Application Priority Data
Mar. 21, 2013 (JP) 2013-058133

(51) Int. Cl. C09K 19/20; C09K 19/38; C09K 19/32; C07C 57/42; C07C 65/28

(52) U.S. Cl. CPC C09K 19/2014; C07C 57/42; C07C 65/28; C09K 19/322; C09K 19/3861; C09K 19/3852; C09K 2019/323

(57) ABSTRACT

The polymerizable composition and the liquid crystal composition containing the polymerizable compound of the present invention have favorable storage stability as evaluated on the basis of the occurrence of precipitation, separation, or the like of crystals during storage. The present invention relates to a polymerizable compound, a liquid crystal composition which contains the compound, and further a liquid crystal display element which contains an optically anisotropic material which is a cured product of the liquid crystal composition, or a cured product which controls alignment of liquid crystal molecules. That is, the present invention relates to a polymerizable compound, and a liquid crystal composition containing the polymerizable compound which contains the polymerizable compound and a liquid crystal compound. The polymerizable compound is a compound represented by General Formula (I), and is useful for an optically anisotropic material, a retardation layer, an alignment film, or a polarizing layer.

### TECHNICAL FIELD

The present invention relates to a polymerizable compound, a liquid crystal composition containing the compound and further a liquid crystal display element containing an optically anisotropic material, which is a cured product of the liquid crystal composition, or a cured product which controls alignment of liquid crystal molecules.

### BACKGROUND ART

Recently, a PSA (Polymer Sustained Alignment) type liquid crystal display, and a PSVA (Polymer Stabilised Vertical Alignment) type liquid crystal display have been developed as a liquid crystal display element in which high speed responsiveness or high contrast can be obtained. In the PSA or the PSVA liquid crystal display element, by performing irradiation with an ultraviolet or the like in a state where a liquid crystal composition containing the polymerizable compound composed of a non-polymerizable liquid crystal composition and a polymerizable compound is disposed between substrates, in a state where, depending on a case, the liquid crystal molecules are aligned by applying a voltage between the substrates, the polymerizable compound is polymerized to store the alignment state of the liquid crystal to the cured product. In addition, in a case where the above is applied to an IPS (In-plane Switching) type liquid crystal display element, it is possible to prepare the display element by curing in a state where the voltage is not applied.

For example, as the related technology, PTL 1 discloses that when the same image is continuously displayed for a long period of time using a liquid crystal compound containing a diacrylate-based polymerizable compound having a biphenyl skeleton, it is possible to reduce an image persistence phenomenon in which a previous image remains visible even if the displayed image is changed.

In addition, recently, the importance of an optical compensation film used for a polarizing plate, a retardation plate, or the like, which is essential for the liquid crystal display has gradually increased, as the information society progresses. An example has been reported in which a polymerizable liquid crystal composition is polymerized and used as the optical compensation film of which high durability and functionality are demanded. (Refer to PTLs 2 to 4).

### CITATION LIST

### Patent Literature

[PTL 1] JP-A-2003-307720

[PTL 2] JP-T-10-513457

[PTL 3] JP-A-2002-145830

[PTL 4] Japanese Patent No. 3948799

### SUMMARY OF INVENTION

### Technical Problem

As described above, in the liquid crystal display element, a problem of reliability such as “image persistence” occurring when the same image is continuously displayed for a long period of time, and problems of storage stability, and productivity caused from the manufacturing process still remain. In particular, the reliability is not a simple problem and is caused by several complex factors. For example, the problem of reliability may be caused by (1) a residual polymerizable compound, (2) a change in inclination of the liquid crystal molecule (a change in pretilt angle), and (3) deterioration of the liquid crystal molecule, or the like, upon irradiation with an ultraviolet ray.

With regard to the reliability, since a polymerization initiator to be used and a decomposed substance thereof cause a decrease in the voltage retention rate of the liquid crystal display element, or an image persistence, a liquid crystal composition containing the polymerizable compound which completes polymerization with a small amount of the ultraviolet ray without using a photopolymerization initiator is demanded. In addition, a change in pretilt angle of the liquid crystal molecule in the liquid crystal composition containing the polymerizable compound is known as the cause of the occurrence of image persistence. That is, in a case where a display element is configured by a soft polymer, which is a cured product of the polymerizable compound, and the same pattern is continuously displayed for a long period of time, a structure of the polymer changes, and as a result, the pretilt angle changes. Since the change in pretilt angle significantly affects response speed, it may cause image persistence. From the above, in order to solve the problem caused by (2), a polymerizable compound which forms a polymer of which the structure is rigid and does not change is effective. However, compatibility with the liquid crystal needs to be improved, because storing the liquid crystal composition at low temperatures becomes difficult. However, when a spacer group is inserted between all ring structures and a polymerizable functional group in order to improve solubility, the rigidity of the molecules is lowered, and the ability to control the inclination of the liquid crystal molecules is degraded.

Therefore, in the liquid crystal display element using the liquid crystal composition containing the polymerizable compound of PTL 1 or the related art, the image persistence properties, solubility, and stability of the pretilt angle were not satisfactory.

In addition, in the optically anisotropic material used for the optical compensation film, or the like, a polymerization velocity of a compound, solubility, a melting point, a glass transition point, transparency of a polymer, mechanical strength, surface hardness, and heat resistance as well as optical properties are important factors. In particular, the optically anisotropic material is useful for a retardation plate of the recent 3D display, and it is considered that it will be widely used in the future. However, for example, in a case where a polymerizable liquid crystal composition is coated on a film substrate such as a triacetyl cellulose (TAC) film and cured, there is a concern that adhesiveness may be reduced, and problems in long term reliability and productivity may occur.

Therefore, an object of the present invention is to provide a liquid crystal display element in which storage stability of the composition when used in the PSA display element, and display properties are improved. In addition, another object of the present invention is to improve adhesiveness of a polymerizable liquid crystal composition when it is coated on a film substrate (for example, triacetyl cellulose (TAC) film, or the like) and cured.

### Solution to Problem

The present inventors have thoroughly studied in order to solve the above described problems, and as a result, found that a polymerizable compound having a specific structure can solve the above described problems, thereby completing the present invention.

The present invention provides a polymerizable compound represented by General Formula (I):

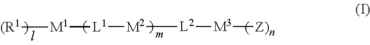

(in General Formula (I), Z represents a hydrogen atom, a C1 to C8 alkyl group, a C1 to C8 halogenated alkyl group, a C1 to C8 alkoxy group, a C1 to C8 halogenated alkoxy group, halogen, a cyano group, a nitro group, or —S¹—R², the S¹ is at least one linking group selected from the group consisting of a single bond and a C1 to C12 alkylene group, one —CH₂— or not-adjacent two or more —CH₂— in the alkylene group may be substituted with —O—, —COO—, —OCO—, or —OCOO—, R¹ and R² each independently represent a hydrogen atom or any one of the following Formulas (R-I) to (R-IX):

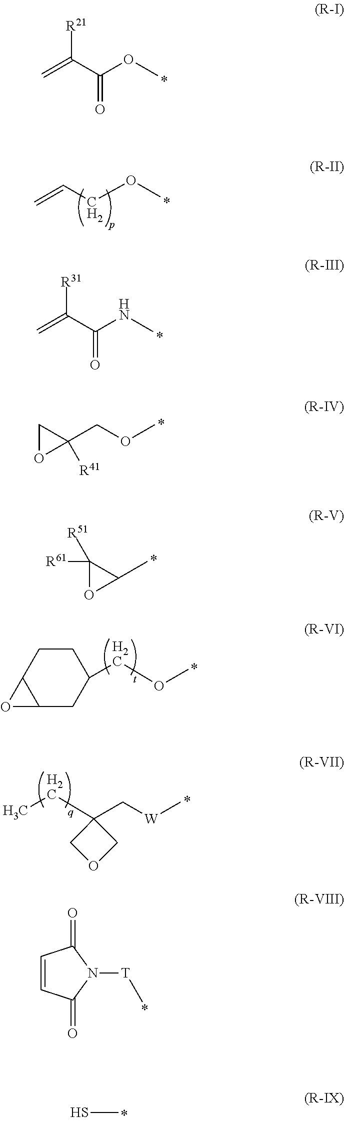

in the formulas (R-I) to (R-IX), R²¹, R³¹, R⁴¹, R⁵¹ and R⁶¹ each independently represent a hydrogen atom, a C1 to C5 alkyl group, or a C1 to C5 halogenated alkyl group, W is a single bond, —O—, or a methylene group, T is a single bond, or —COO—, p, t, and q each independently are 0, 1, or 2,

L¹ and L² each independently represent a single bond, —O—, —S—, —CH₂—, —OCH₂—, —CH₂O—, —CO—, —C₂H₄—, —COO—, —OCO—, —OCOOCH₂—, —CH₂OCOO—, —OCH₂CH₂O—, —CO—NR^{a}—, —NR^{a}—CO—, —SCH₂—, —CH₂S—, —CH═CR^{a}—COO—, —CH═CR^{a}—OCO—, —COO—CR^{a}═CH—, —OCO—CR^{a}═CH—, —COO—CR^{a}═CH—COO—, —COO—CR^{a}═CH—OCO—, —OCO—CR^{a}═CH—COO—, —OCO—CR^{a}═CH—OCO—, —COOC₂H₄—, —OCOC₂H₄—, —C₂H₄OCO—, —(CH₂)ⱼ—C(═O)—O—, —(CH₂)ⱼ—O—(C═O)—, —O—(C═O)—(CH₂)ⱼ—, —(C═O)—O—(CH₂)ⱼ—, —CH₂OCO—, —COOCH₂—, —OCOCH₂—, —CH═CH—, —CF═CF—, —CF═CH—, —CH═CF—, —CF₂—, —CF₂O—, —OCF₂—, —CF₂CH₂—, —CH₂CF₂—, —CF₂CF₂—, or —C≡C— (in the formulas, R^{a}s each independently represent a hydrogen atom or a C1 to C4 alkyl group, and in the formulas, j represents an integer of 1 to 4),

M¹ and M³ each independently represent an aromatic ring or an aliphatic ring, M² represents a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, or a 1,3-dioxane-2,5-diyl group, the M¹, M², and M³ each independently may not be substituted or may be substituted with a C1 to C8 alkyl group, a C1 to C8 halogenated alkyl group, a C1 to C8 alkoxy group, halogen, a cyano group, or a nitro group,

l and n each independently represent an integer of 0 to 4, and satisfy 1+n≧1 (provided that, when 1 represents 0, R¹ is a hydrogen atom, and Z has any one group of Formulas (R-I) to (R-IX), and when n represents 0, R¹ has any one group of Formulas (R-I) to (RIX))

m represents an integer of 1 to 4, and when m is equal to or more than 2, when m is more than 2, L1 and M2 are same or may be different, but at least one of L¹ represents a single bond)

The present invention provides a polymerizable composition which contains the polymerizable compound, a liquid crystal composition containing the polymerizable compound which contains the polymerizable compound, an optically anisotropic material configured by a polymer of the liquid crystal composition containing the polymerizable compound, the liquid crystal composition containing the polymerizable compound which contains the polymerizable compound and the non-polymerizable liquid crystal compound, and a liquid crystal display element which uses the liquid crystal composition containing the polymerizable compound to which a liquid crystal alignment ability is imparted by polymerizing the polymerizable compound in the liquid crystal composition containing the polymerizable compound.

### Advantageous Effects of Invention

The optically anisotropic material using the polymerizable compound or the composition containing the polymerizable compound of the present invention has favorable adhesiveness to substrates, and is useful for a polarizing plate, a retardation plate, or the like.

In the present invention, in a case where the liquid crystal display element to which a liquid crystal alignment ability is imparted by polymerizing the polymerizable compound in the liquid crystal composition containing the polymerizable compound is used, the polymerizable compound can be polymerized by light or heat without adding a polymerization initiator, or adding a trace amount of the polymerization initiator, and impurities derived from the photoinitiator do not affect or minimally affect the liquid crystal display element. Thus, it is possible to obtain both reliability and productivity. It is possible to provide a liquid crystal display element in which the stability of the pretilt angle is greatly improved compared to the related art, by using the polymerizable compound of the present invention.

The polymerizable composition and the liquid crystal composition containing the polymerizable compound of the present invention have preferable storage stability as evaluated on the basis of the occurrence of precipitation, separation, or the like of crystals during storage.

### DESCRIPTION OF EMBODIMENTS

Hereinafter, embodiments of the present invention will be described in detail. In addition, the present application is based on the Japanese Patent Application no. 2013-058133 filed on Mar. 21, 2013, and the entire contents of which are cited herein by reference.

A first aspect of the present invention is a polymerizable compound represented by General Formula (I):

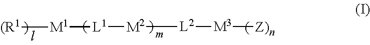

(in General Formula (I), Z represents a hydrogen atom, a C1 to C8 alkyl group, a C1 to C8 halogenated alkyl group, a C1 to C8 alkoxy group, a C1 to C8 halogenated alkoxy group, halogen, a cyano group, a nitro group, or —S¹—R², the S¹ is at least one linking group selected from the group consisting of a single bond and a C1 to C12 alkylene group, one —CH₂— or not-adjacent two or more —CH₂— in the alkylene group may be substituted with —O—, —COO—, —OCO—, or —OCOO—, R¹ and R² each independently represent a hydrogen atom or any one of the following Formulas (R-I) to (R-IX):

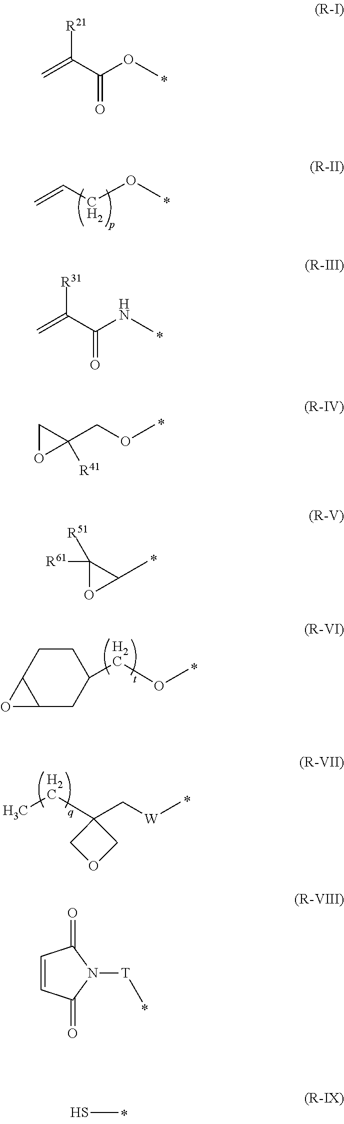

in the formulas (R-I) to (R-IX), R²¹, R³¹, R⁴¹, R⁵¹ and R⁶¹ each independently represent a hydrogen atom, a C1 to C5 alkyl group, or a C1 to C5 halogenated alkyl group, W is a single bond, —O—, or a methylene group, T is a single bond, or —COO—, p, t, and q each independently are 0, 1, or 2,

L¹ and L² each independently represent a single bond, —O—, —S—, —CH₂—, —OCH₂—, —CH₂O—, —CO—, —C₂H₄—, —COO—, —OCO—, —OCOOCH₂—, —CH₂OCOO—, —OCH₂CH₂O—, —CO—NR^{a}—, —NR^{a}—CO—, —SCH₂—, —CH₂S—, —CH═CR^{a}—COO—, —CH═CR^{a}—OCO—, —COO—CR^{a}═CH—, —OCO—CR^{a}═CH—, —COO—CR^{a}═CH—COO—, —COO—CR^{a}═CH—OCO—, —OCO—CR^{a}═CH—COO—, —OCO—CR^{a}═CH—OCO—, —COOC₂H₄—, —OCOC₂H₄—, —C₂H₄OCO—, —(CH₂)ⱼ—C(═O)—O—, —(CH₂)ⱼ—O—(C═O)—, —O—(C═O)—(CH₂)ⱼ—, —C═O)—O—(CH₂)ⱼ—, —CH₂OCO—, —COOCH₂—, —OCOCH₂—, —CH═CH—, —CF═CF—, —CF═CH—, —CH═CF—, —CF₂—, —CF₂O—, —OCF₂—, —CF₂CH₂—, —CH₂CF₂—, —CF₂CF₂—, or —C≡C— (in the formulas, R^{a}s each independently represent a hydrogen atom or a C1 to C4 alkyl group, and in the formulas, j represents an integer of 1 to 4),

M¹ and M³ each independently represent an aromatic ring or an aliphatic ring, M² represents a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, or a 1,3-dioxane-2,5-diyl group, the M¹, M², and M³ each independently may not be substituted or may be substituted with a C1 to C8 alkyl group, a C1 to C8 halogenated alkyl group, a C1 to C8 alkoxy group, halogen, a cyano group, or a nitro group,

l and n each independently represent an integer of 0 to 4, and satisfy 1+n≧1 (provided that, when 1 represents 0, R¹ is a hydrogen atom, and Z has any one group of Formulas (R-I) to (R-IX), and when n represents 0, R¹ has any one group of Formulas (R-I) to (RIX))

m represents an integer of 1 to 4, and when m is equal to or more than 2, L¹ and M², of which there are two each, may be the same as each other or different from each other, but at least one of L¹ represents a single bond.)

Since the polymerizable compound of the present invention has the chemical structure of General Formula (I) described above, the compound has rigidity, and an ultraviolet ray absorbing region on the long wavelength side is extended, thereby exhibiting an effect of promoting hardenability.

In General Formula (I) according to the present invention, Z represents a hydrogen atom, a C1 to C8 alkyl group, a C1 to C8 halogenated alkyl group, a C1 to C8 alkoxy group, a C1 to C8 halogenated alkoxy group, halogen, a cyano group, a nitro group, or —S¹—R², the S¹ represents at least one linking group selected from the group consisting of a single bond and a C1 to C12 alkylene group (one —CH₂— or not-adjacent two or more —CH₂— in the alkylene group may be substituted with —O—, —COO—, —OCO—, or —OCOO—), when the compound is used for the display element, Z is preferably —S¹—R², S¹ is more preferably C1 to C12 alkylene group or a single bond, and particularly preferably a single bond.

Since a polymer formed from this polymerizable compound forms a polymer of which a structure does not change and is rigid, change in pretilt is suppressed, and it is optimal for the PSA, and PSVA liquid crystal display element.

In General Formula (I) according to the present invention, R¹ and R² in (—S¹—R²), which is one aspect of Z, each independently represent a polymerizable group, the R¹ and R² are one polymerizable group selected from the group consisting of Formulas (R-I) to (R-IX) described above, and more specific examples of the polymerizable group include the structures described below.

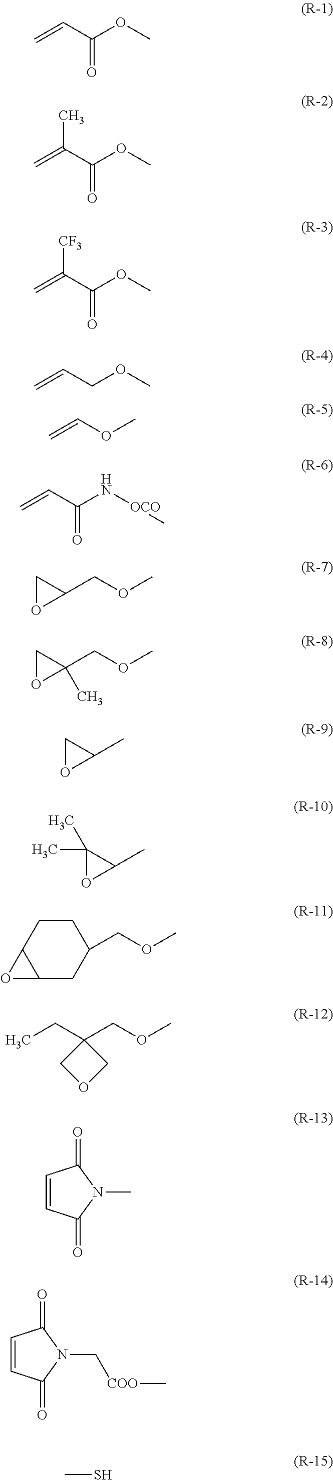

These polymerizable groups are cured by radical polymerization, radical addition polymerization, cation polymerization, and anion polymerization. In particular, in a case where an ultraviolet ray polymerization is performed as a polymerization method, Formula (R-1), Formula (R-2), Formula (R-4), Formula (R-5), Formula (R-7), Formula (R-11), Formula (R-13), or Formula (R-15) is preferable, Formula (R-1), Formula (R-2), Formula (R-7), Formula (R-11), or Formula (R-13) is more preferable, and Formula (R-1), or Formula (R-2) is still more preferable.

In addition, in General Formula (I) described above, R¹ is a Formula (R-2), and R² particularly preferably represents a Formula (R-1).

In General Formula (I) according to the present invention, L¹ and L² each independently are a single bond, —O—, —S—, —CH₂—, —OCH₂—, —CH₂O—, —CO—, —C₂H₄—, —COO—, —OCO—, —OCOOCH₂—, —CH₂OCOO—, —OCH₂CH₂O—, —CO—NR^{a}—, —NR^{a}—CO—, —SCH₂—, —CH₂S—, —CH═CR^{a}—COO—, —CH═CR^{a}—OCO—, —COO—CR^{a}═CH—, —OCO—CR^{a}═CH—, —COO—CR^{a}═CH—COO—, —COO—CR^{a}═CH—OCO—, —OCO—CR^{a}═CH—COO—, —OCO—CR^{a}═CH—OCO—, —(CH₂)ⱼ—C(═O)—O—, —(CH₂)ⱼ—O— (C═O)—, —O— (C═O)—(CH₂)ⱼ—, —(C═O)—O—(CH₂)ⱼ—, —CH═CH—, —CF═CF—, —CF═CH—, —CH═CF—, —CF₂—, —CF₂O—, —OCF₂—, —CF₂CH₂—, —CH₂CF₂—, —CF₂CF₂—, or —C≡C— (in the formulas, R^{a}s each independently represent a hydrogen atom or a C1 to C4 alkyl group, in the formulas, j represents an integer of 1 to 4). In addition, the L¹ is preferably at least one selected from the group consisting of a single bond, —O—, —S—, —OCH₂—, —CH₂O—, —CO—, —C₂H₄—, —COO—, —OCO—, —OCOOCH₂—, —CH₂OCOO—, —CH═CH—COO—, —OCO—CH═CH—, —COO—CH═CH—, —CH═CH—OCO—, —(CH₂)ⱼ—C(═O)—O—, —(CH₂)ⱼ—O—(C═O)—, —O—(C═O)—(CH₂)ⱼ—, —(C═O)—O—(CH₂)ⱼ—, —CH═CH—, —CF₂—, —CF₂O—, —OCF₂—, —CF₂CH₂—, —CH₂CF₂—, —CF₂CF₂—, and —C≡C— (in the formulas, j represents an integer of 1 to 4), and the L² is preferably a single bond, —OCH₂CH₂O—, —(CH₂)ⱼ—C(═O)—O—, —(CH₂)ⱼ—O—(C═O)—, —O—(C═O)—(CH₂)ⱼ—, or —(C═O)—O—(CH₂)ⱼ—. Further, the L¹ is preferably a single bond, —OCH₂—, —CH₂O—, —C₂H₄—, —COO—, —OCO—, —CH═CH—COO—, —OCO—CH═CH—, —COO—CH═CH—, —CH═CH—OCO—, —COOC₂H₄—, —OCOC₂H₄—, —C₂H₄OCO—, —C₂H₄COO—, —CF₂O—, —OCF₂—, and —C≡C—, and is more preferably a single bond, —COO—, —OCO—, —OCH₂—, or —CH₂O— from a viewpoint of manufacturing at a low cost, and the alignment properties of liquid crystals. However, at least one of plural L¹s preferably represents a single bond. Meanwhile, the L² is preferably —OCOC₂H₄— or —COOC₂H₄— from a viewpoint of solubility, and increasing the wavelength of the ultraviolet ray absorbing region.

Further, one of L¹ and L² is preferably a single bond from a viewpoint of reliability.

In General Formula (I) according to the present invention, M² is at least one selected from the group consisting of a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, and a 1,3-dioxane-2,5-diyl group, and is preferably a 1,4-phenylene group, a 1,4-cyclohexylene group or a naphthalene-2,6-diyl group.

In General Formula (I) according to the present invention, M¹ and M³ each independently are an aromatic ring or an aliphatic ring, preferably a divalent to tetravalent aromatic ring or aliphatic ring, and more specifically, any one of the following Formulas (i) to (xxvi):

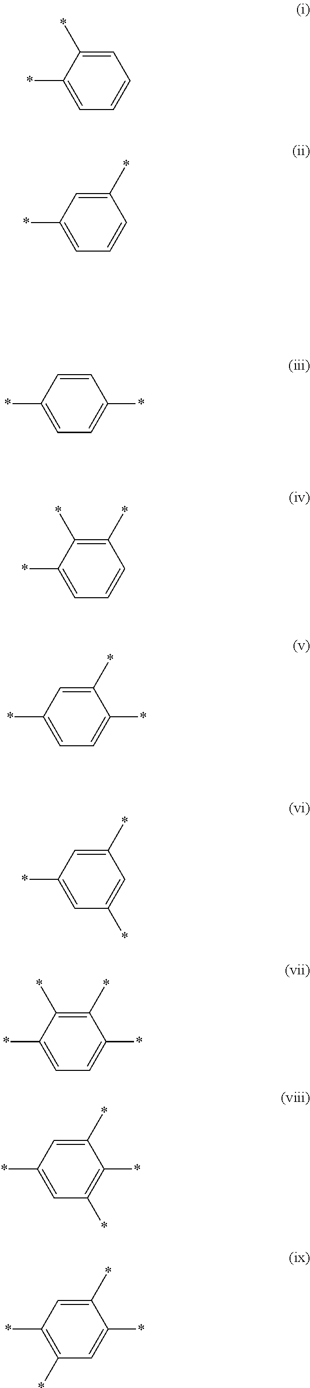

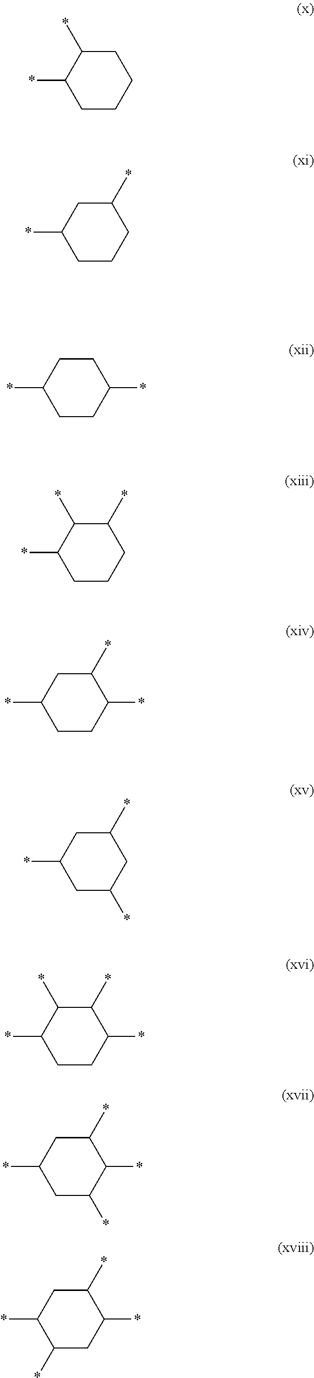

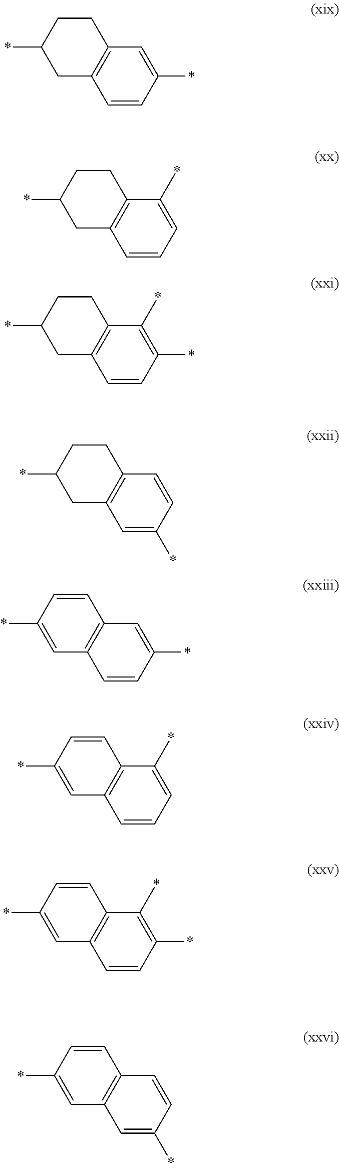

(* in the Formulas (i) to (xxvi) described above represents a binding site.) is preferable. Among the above, M³ in General Formula (I) according to the present invention is more preferably a 1,3,5-benzenetriyl group (Formula (vi)), a 1,3,4-benzenetriyl group (Formula (v)), a 1,3,4-cyclohexanetriyl group (Formula (xiv)), or a 1,3,5-cyclohexanetriyl group (Formula (xv)), in particular, the 1,3,4-benzenetriyl group (Formula (v)) is preferable. Meanwhile, M¹ in General Formula (I) according to the present invention, in particular, is preferably a 1,4-phenylene group, a pyridine-2,5-diyl group, a pyrimidine-2,5-diyl group, a naphthalene-2,6-diyl group or a 1,3,4-benzenetriyl group.

In General Formula (I) according to the present invention, 1 and n each independently represent an integer of 0 to 4, and satisfy 1+n≧1 (provided that, when 1 represents 0, R¹ is a hydrogen atom, and Z has any one group of Formulas (R-I) to (R-IX), and when n represents 0, R¹ has any one group of Formulas (R-I) to (RIX). In addition, 1+n≧2 is preferable, and, 1+n≧3 is more preferable.

l is preferably an integer of 1 to 3, and more preferably an integer of 1 to 2. n is preferably an integer of 1 to 3, more preferably 2 or 3, and particularly preferably 2.

When l is from 1 to 2, it is preferable from a viewpoint of increasing the elastic modulus of the polymer. When n is from 1 to 3, it is preferable from a viewpoint of improving solubility.

In General Formula (I) according to the present invention, m represents an integer of 1 to 4, when m is equal to or more than 2, L¹ and M², of which there are two each, may be the same as each other or different from each other, and at least one of L¹ represents a single bond. In addition, m in the formula is preferably 1 or 2, and particularly preferably 1.

One example of a preferred aspect of the compound represented by General Formula (I) according to the present invention is General Formula (Ia):

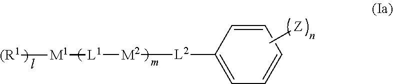

(In General Formula (Ia), Z is independently —S¹—R² (the R² is at least one selected from the group consisting of Formula (R-1) to Formula (R-15)), l and n each independently represent an integer of 1 to 3, and satisfy 1+n≧2, and in General Formula (Ia), since R¹, M¹, L¹, M², L², m, and S¹ are the same as those in General Formula (I), they are omitted).

In addition, in the compound represented by General Formula (Ia), m is preferably an integer of 1 to 2.

In addition, one example of a preferred aspect of General Formula (Ia) is General Formula (Ib):

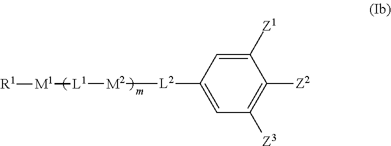

(in General Formula (Ib), Z¹, Z² and Z³ each independently are at least one selected from the group consisting of a hydrogen atom and Formula (R-I) to Formula (R-IX), and in General Formula (Ia), since R¹, M¹, L¹, M², L², m, and S¹ are the same as those in General Formula (I), they are omitted).

Further, a particularly preferable aspect of the polymerizable compound according to the present invention is an aspect in which in General Formula (Ib), m is equal to or more than 1, R¹ is at least one selected from the group consisting of Formula (R-I) to Formula (R-IX), at least two groups of Z¹, Z² and Z³ are the —S¹—R² (R² is any one of Formula (R-I) to Formula (R-IX)), L¹ or L² is the same as that in General Formula (I), but any one of L¹ and L² is —(CH₂)_{z}—C(═O)—O—, —(CH₂)z-O—(C═O)—, —O—(C═O)—(CH₂)_{z}—, or —(C═O)—O—(CH₂) z-, M² represents a 1,4-phenylene group, a 1,4-cyclohexylene group, a pyridine-2,5-diyl group, a pyrimidine-2,5-diyl group, a naphthalene-2,6-diyl group, an indane-2,5-diyl group, a 1,2,3,4-tetrahydronaphthalene-2,6-diyl group, or a 1,3-dioxane-2,5-diyl group, which is not substituted, or substituted with at least one group selected from the group consisting of a C1 to C12 alkyl group, a C1 to C12 halogenated alkyl group, a C1 to C12 alkoxy group, a C1 to C12 halogenated alkoxy group, halogen, a cyano group, and a nitro group, and M¹ is any one of following General Formulas (1-1) or (1-2):

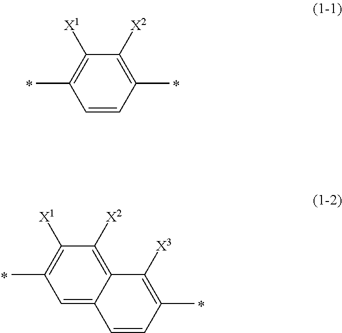

(in General Formula (1-1) and General Formula (1-2), X¹, X² and X³ each independently are at least one selected from the group consisting of a hydrogen atom, a halogen atom, a C1 to C15 alkoxy group, and —OCO(C_{w}H_{2w+1}); in the formula, w is an integer of 1 to 5).

When the polymerizable compound having this chemical structure is added, for example, to the liquid crystal composition, it is possible to produce a rigid polymer having a high crosslinking density as well as excellent compatibility with other non-polymerizable liquid crystal compounds. Therefore, it is possible to strongly maintain the alignment regulation force of the coexisting liquid crystal compound. In addition, when the polymerizable compound according to the present invention has three or more ring structures, because the liquid crystal composition containing the polymerizable compound has three or more ring structures, it is possible to rapidly perform a polymerization reaction by efficiently absorbing light energy.

More specifically, the compound represented by General Formula (I) according to the present invention is preferably at least one selected from the group consisting of compounds represented by General Formulas (I-1) to (I-44).

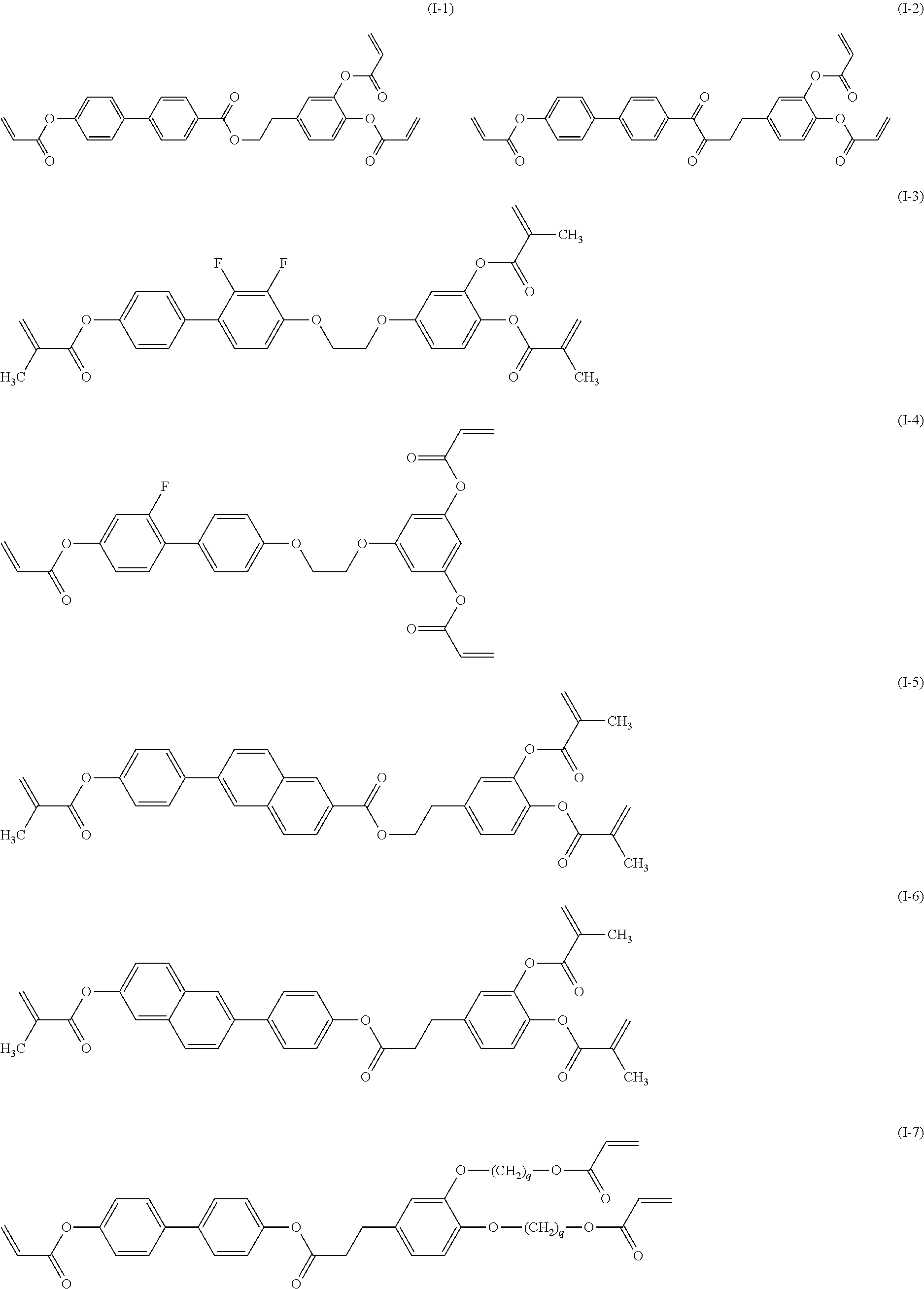

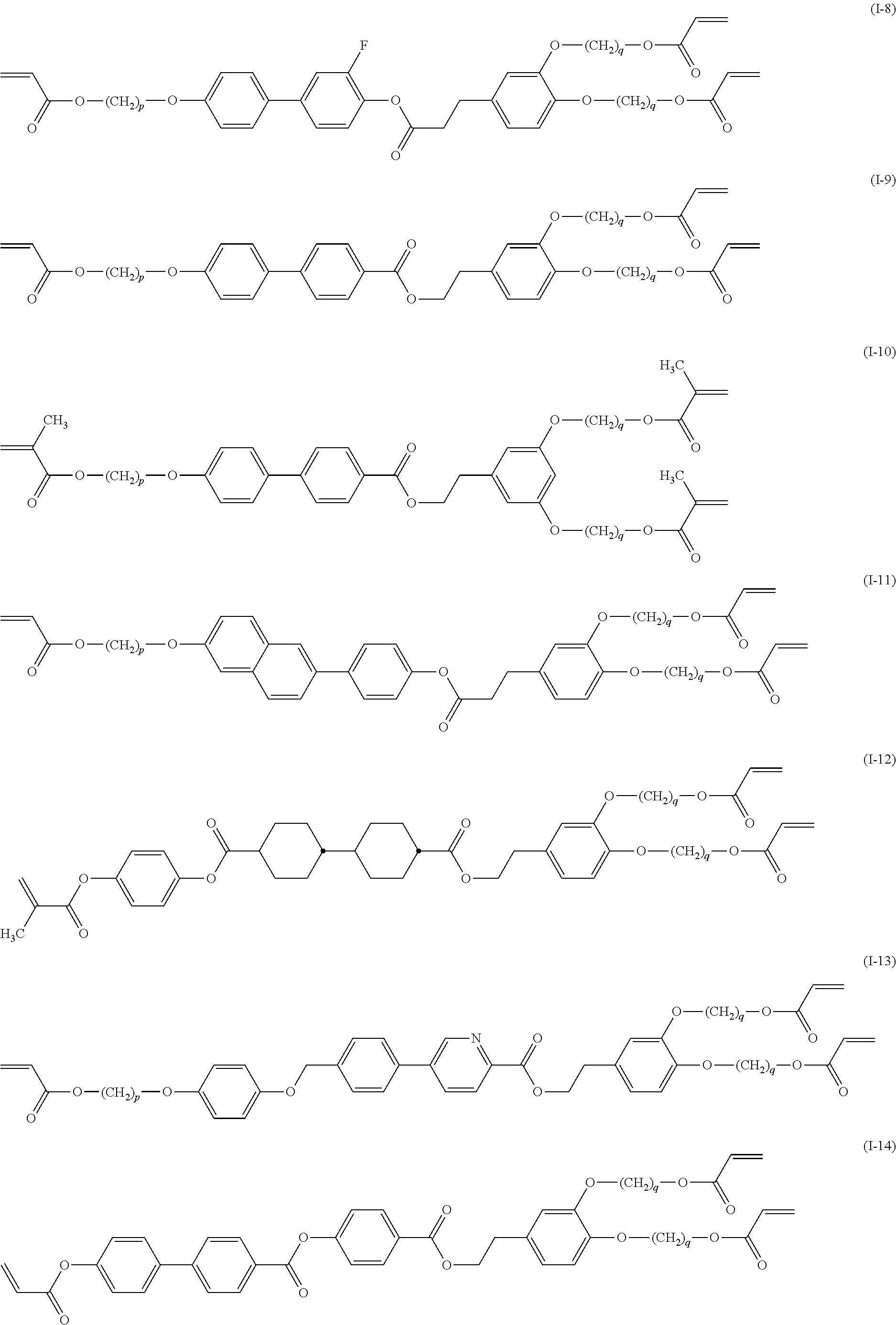

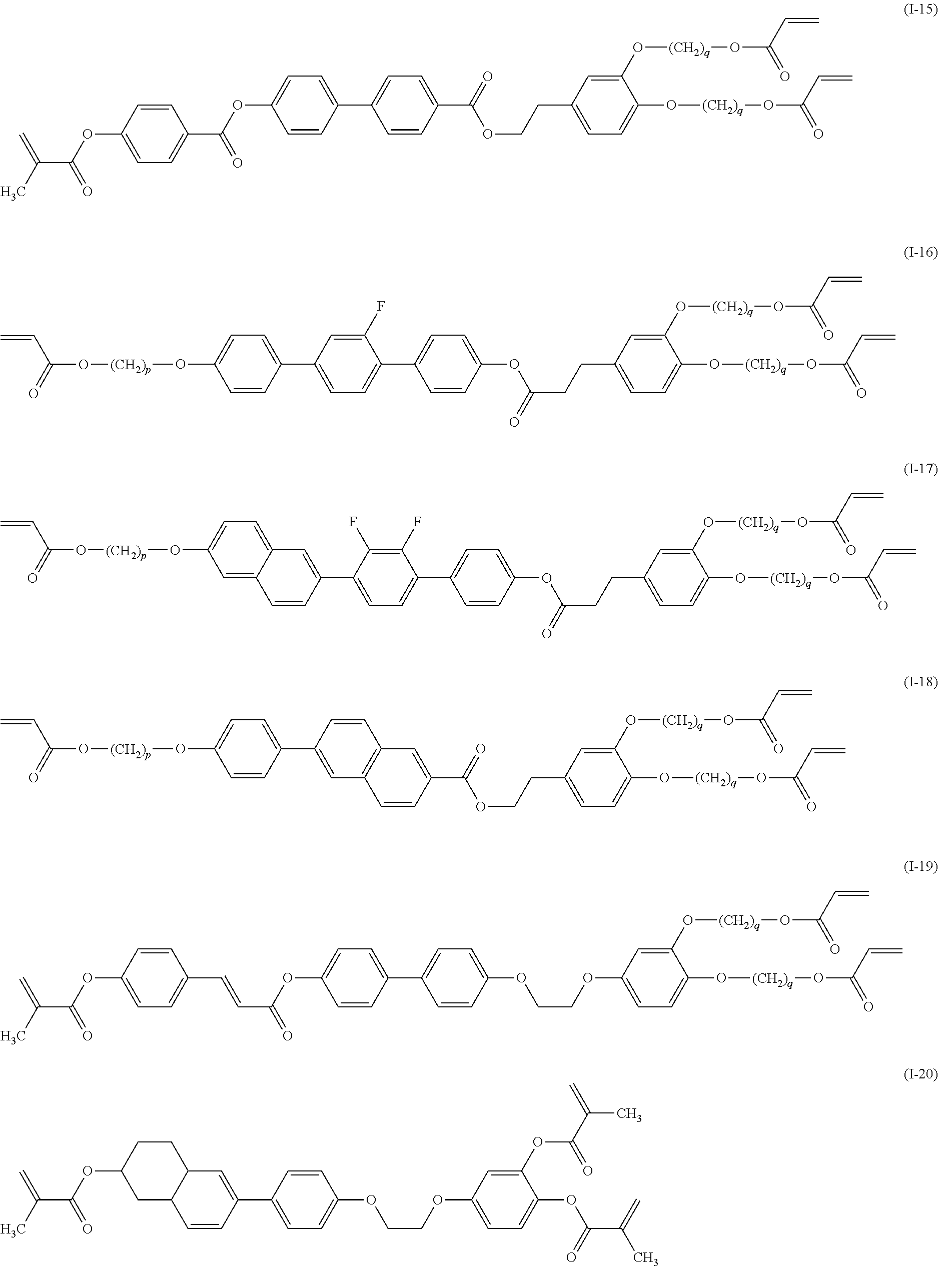

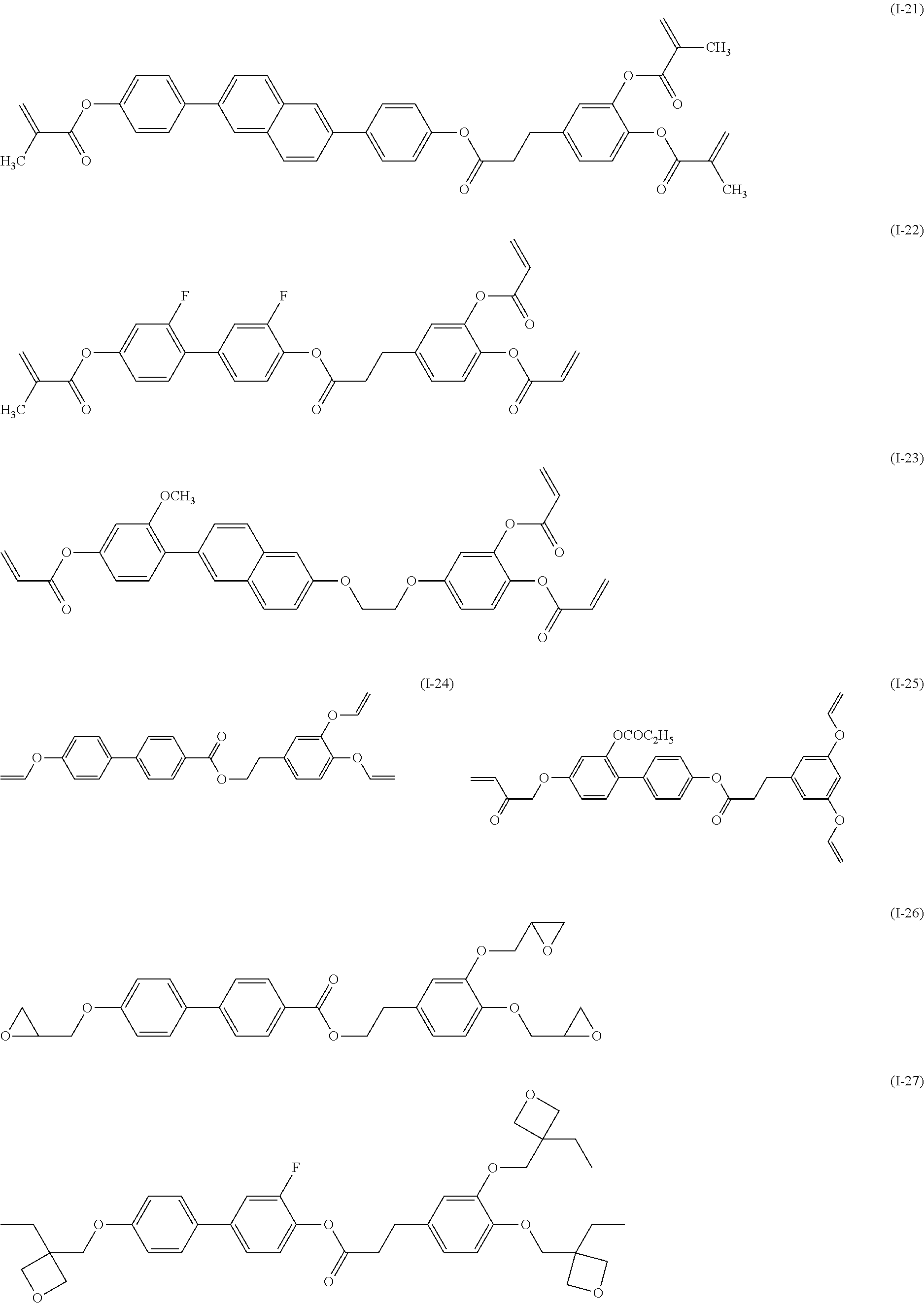

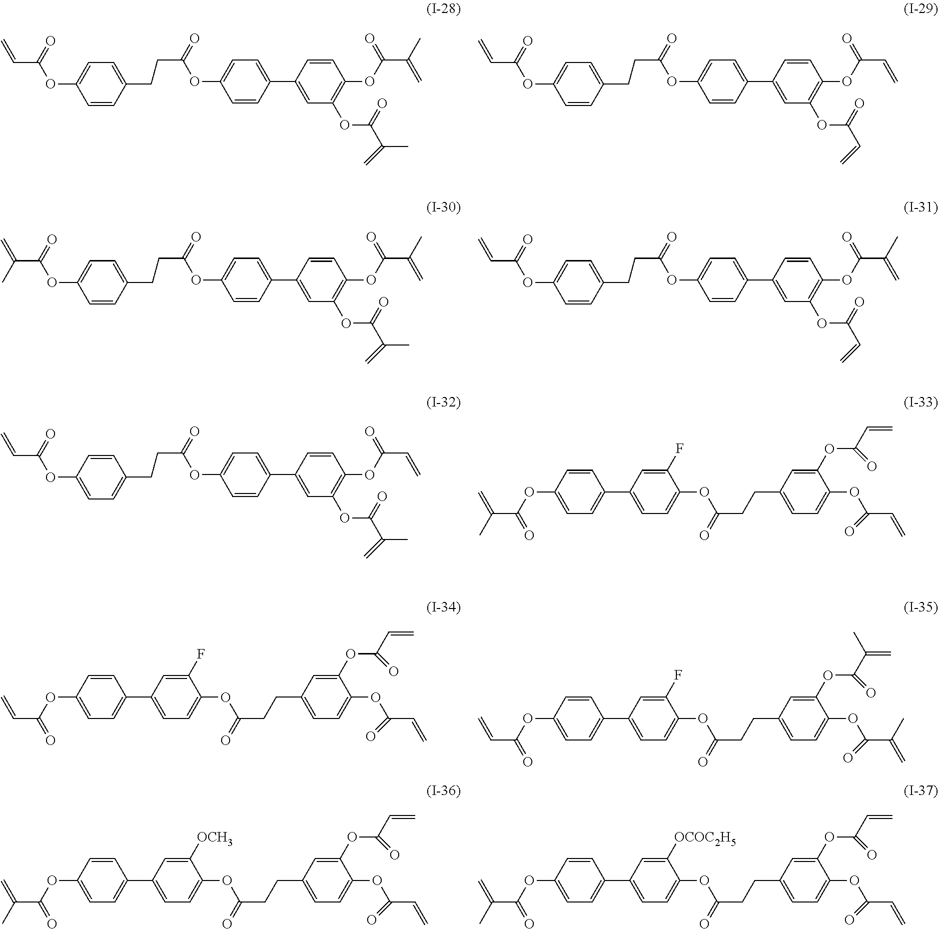

(In the formula, p and q represent an integer of 0 to 12, and when p is 0 and oxygen atoms are directly bonded to each other, one oxygen atom is removed.)

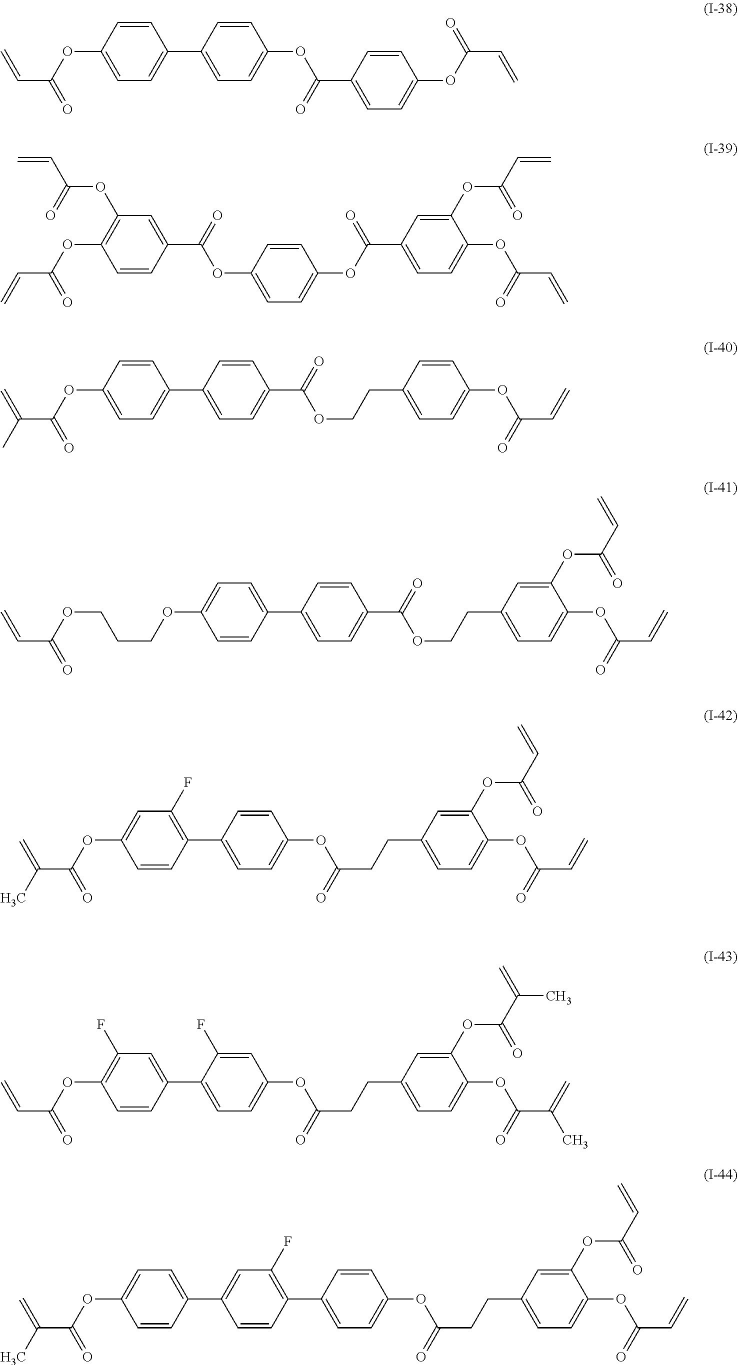

The polymerizable compound of the present invention can be synthesized by the synthesis methods described below.

(Preparation Method 1) Preparation of Compound Represented by General Formula (I-2)

Biphenol and a caffeic acid in which a phenolic hydroxyl group is protected by a tetrahydropyranyl group are esterified and a catalytic hydrogen reduction is carried out to obtain a biphenol derivative (S-1) having a biphenyl skeleton. Further, the protecting group is eliminated using a hydrochloric acid, and an esterification reaction of the biphenol derivative (S-1) and acryloyl chloride is carried out to obtain an object substance (I-2).

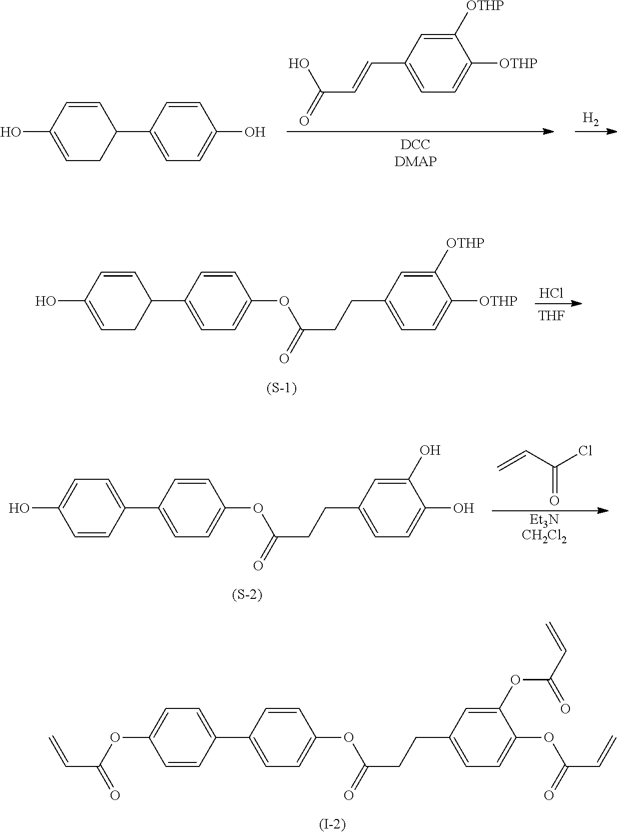

(Preparation Method 2) Preparation of Compound Represented by General Formula (I-4)

The Mitsunobu reaction of 4,4′-dihydroxy-3-fluorobiphenyl and ethylene glycol mono tertiary butyl ether is carried out using triphenyl phosphine and a diisopropyl azodicarboxylic acid to obtain a biphenol derivative (S-3), and further an esterification reaction of the biphenol derivative (S-3) and acryloyl chloride is carried out to obtain an acrylic acid derivative (S-4). Next, a tertiary butyl group is eliminated by a trifluoroacetic acid and is converted to ethanol to obtain an acrylic acid derivative (S-5).

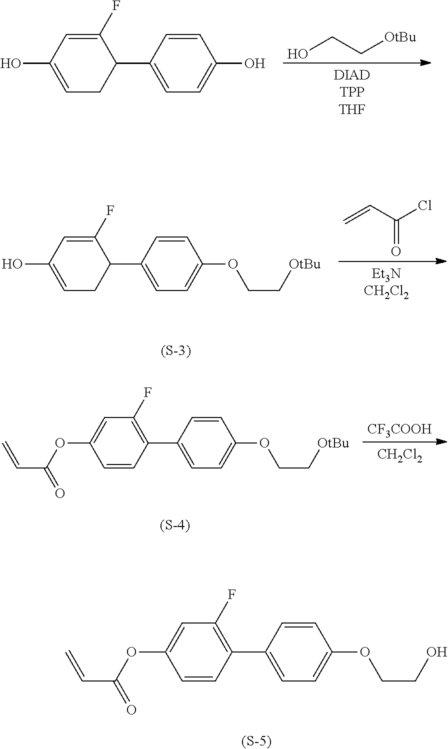

Next, the Mitsunobu reaction of the acrylic acid derivative (S-5) and 3,5-diacryloxyphenol is carried out using triphenyl phosphine and a diisopropyl azodicarboxylic acid to obtain an object compound (I-4).

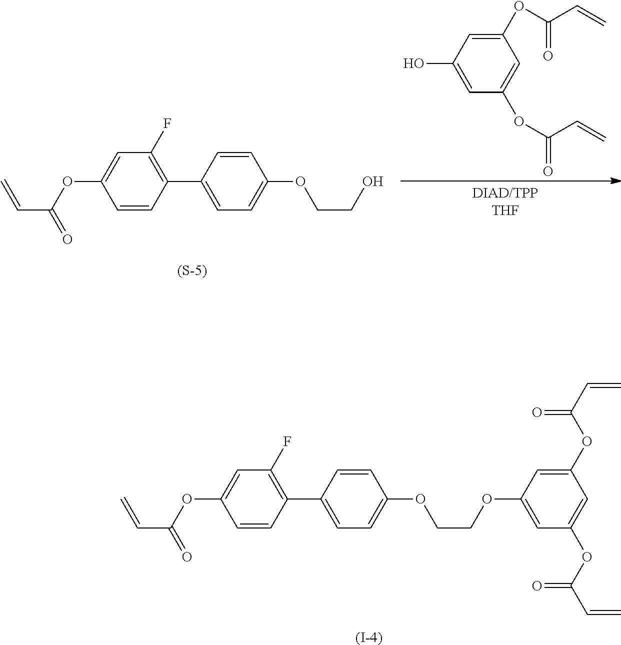 [SMILES: C=CC(=O)Oc1ccc(-c2ccc(OCCO)cc2)c(F)c1 ; C=CC(=O)Oc1cc(O)cc(OC(=O)C=C)c1 ; "(S-5)" ; C=CC(=O)Oc1cc(OCCOc2ccc(-c3ccc(OC(=O)C=C)cc3F)cc2)cc(OC(=O)C=C)c1 ; "(I-4)"]

(Preparation Method 3) Preparation of Compound Represented by General Formula (I-6)

The Suzuki coupling reaction of 2-bromo-6-oxytetrahydroxypyranyl naphthalene and a hydroxyphenyl boric acid is carried out to obtain a phenylnaphthalene derivative (S-6), further it is esterified with a caffeic acid in which a phenolic hydroxyl group is protected by a tetrahydropyranyl group, and a catalytic hydrogen reduction is carried out to obtain a phenylnaphthalene derivative (S-7). Next, a phenol protecting group is eliminated using a hydrochloric acid to obtain a naphthol derivative (S-8).

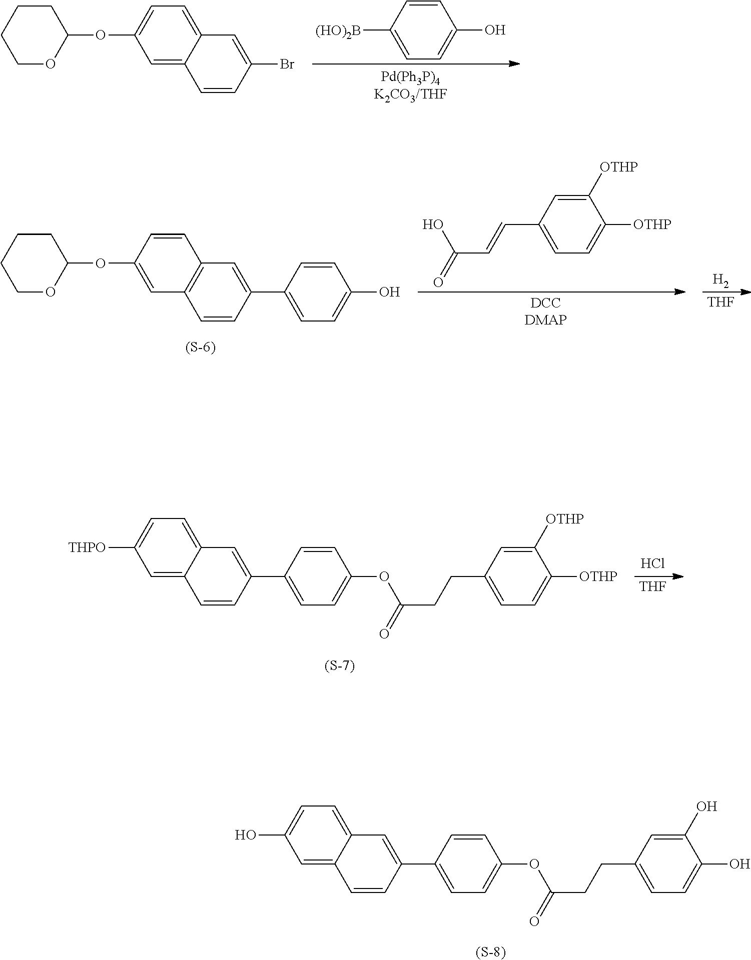

Next, an object compound (I-6) is obtained by an esterification reaction using methacryloyl chloride.

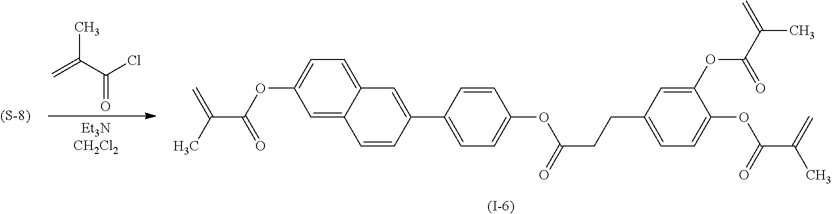

(Preparation Method 4) Preparation of Compound Represented by General Formula (I-12)

An esterification reaction of 4-methacryloyloxyphenol and trans-trans-4,4′-bicyclohexanedicarboxylic acid monotertiarybutylester is carried out using a dehydration condensation agent such as dicyclohexylcarbodiimide to obtain a bicyclohexane derivative (S-9). Further, a tertiary butyl group is eliminated using a trifluoroacetic acid to obtain a bicyclohexanecarboxylic acid derivative (S-10).

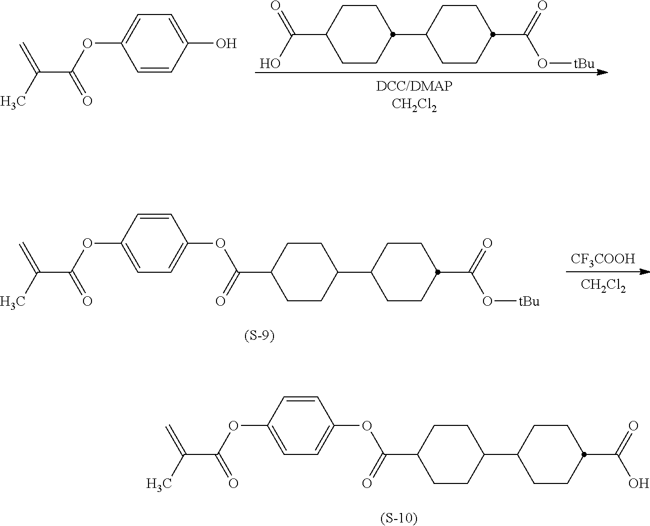

Next, an etherification reaction of 3,4-dihydroxyphenyl ethanol and 6-chlorohexyl acrylate is carried out using a base such as potassium carbonate to obtain acrylate (S-11) having a hydroxyl group. After that, an esterification reaction of the acrylate (S-11) and the (S-10) is carried out using a dehydration condensation agent such as dicyclohexylcarbodiimide to obtain an object compound (I-12).

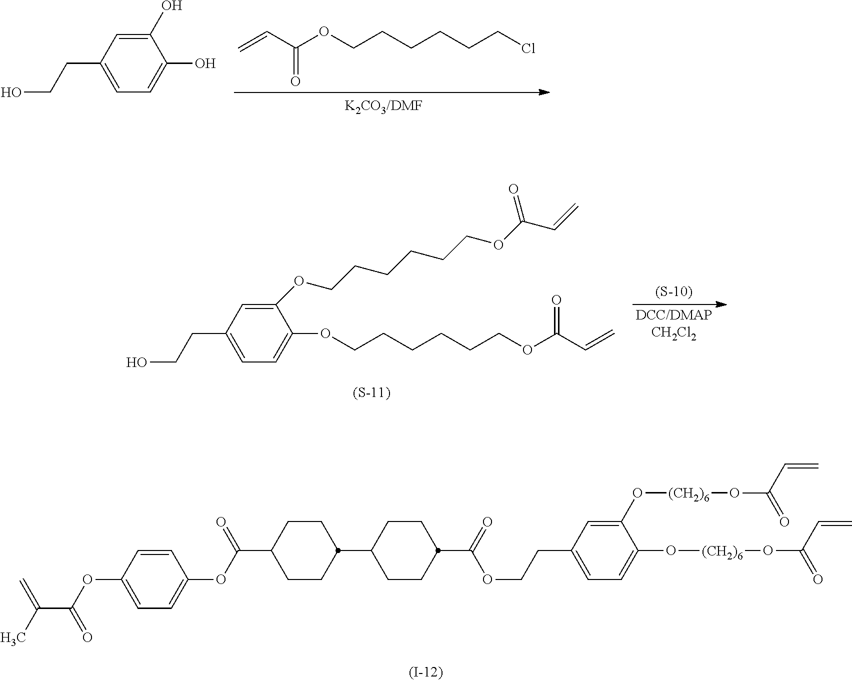

(Preparation Method 5) Preparation of Compound Represented by General Formula (I-19)

The Mitsunobu reaction of 4,4′-hydroxybiphenyl and ethylene glycol mono tertiary butyl ether is carried out using triphenyl phosphine and a diisopropyl azodicarboxylic acid to obtain a biphenol derivative (S-12), and further an esterification reaction of the biphenol derivative (S-12) and a p-acryloyloxycinnamic acid is carried out using a dehydration condensation agent such as dicyclohexylcarbodiimide to obtain a biphenol derivative (S-13) having a methacryloyl group. Next, a tertiary butyl group is eliminated using a trifluoroacetic acid and is converted to ethanol to obtain a methacrylate derivative (S-14).

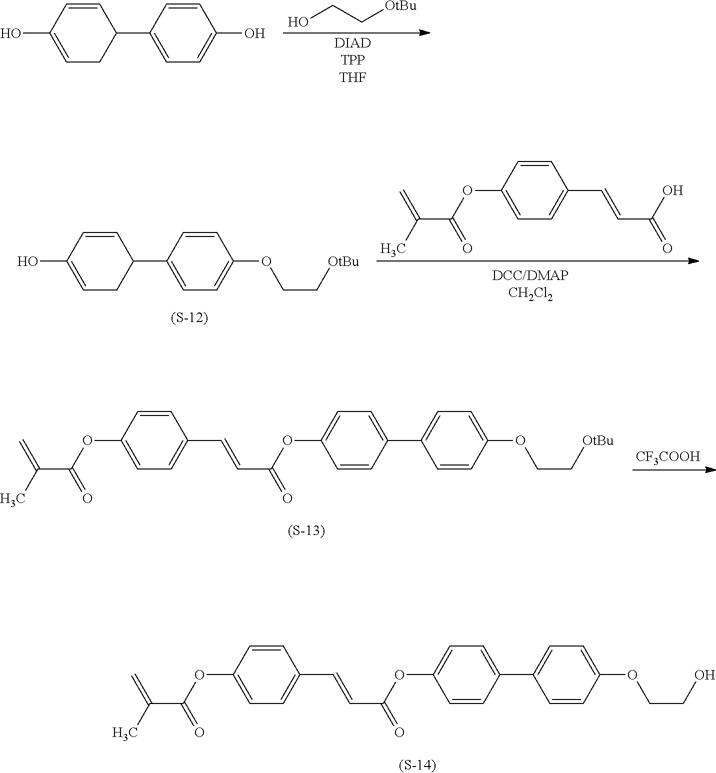

Next, the Mitsunobu etherification reaction of the methacrylate derivative (S-14) and 3,4-(4-acryloyloxybutoxy)phenol is carried out using triphenyl phosphine and a diisopropyl azodicarboxylic acid to obtain an object compound (I-19).

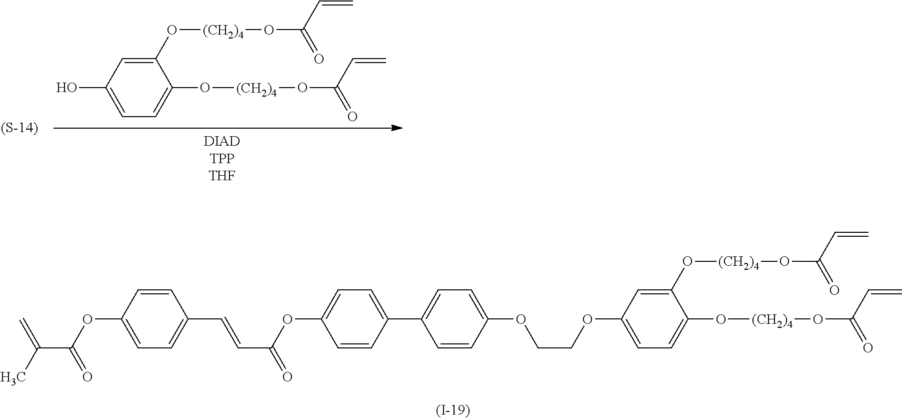

(Preparation Method 6) Preparation of Compound Represented by General Formula (I-24)

A transesterification reaction of ethyl 4-(4-hydroxybiphenyl)benzoate and 3,4-dihydroxyphenyl ethanol is carried out using a tin catalyst to obtain a phenol derivative (S-15) having a biphenyl skeleton. Further, the Mitsunobu etherification reaction of the phenol derivative (S-15) and vinyl alcohol is carried out using triphenyl phosphine and a diisopropyl azodicarboxylic acid to obtain an object substance (I-24).

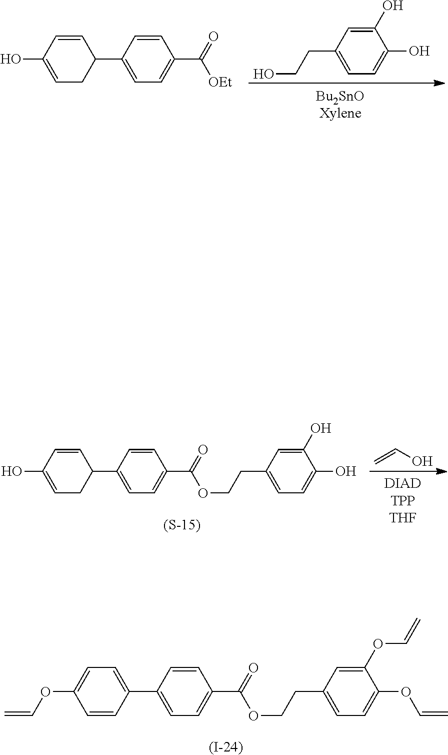

(Preparation Method 7) Preparation of Compound Represented by General Formula (I-27)

The Suzuki coupling reaction of 4-bromo-2-fluorophenol and a 4-tetrahydropyranyloxyphenyl boric acid is carried out to obtain a biphenyl derivative (S-16).

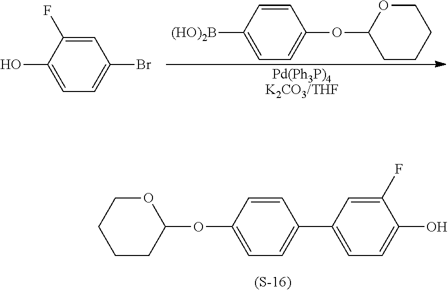

A phenolic hydroxyl group of caffeic acid ethyl ester is protected by 3,4-dihydro-2H-pyran, and further a catalytic hydrogen reduction is carried out to obtain a catechol derivative (S-17). Next, hydrolysis is carried out using sodium hydroxide to obtain a propionic acid derivative (S-18). An esterification reaction of the propionic acid derivative (S-18) and the biphenyl derivative (S-16) is carried out using a dehydration condensation agent such as dicyclohexylcarbodiimide to obtain a catechol derivative (S-19), and further a tetrahydropyranyl group is eliminated using a hydrochloric acid to obtain a catechol derivative (S-20). Next, the Mitsunobu reaction of the catechol derivative (S-20) and 3-ethyl-3-oxetanemethanol is carried out using triphenyl phosphine and a diisopropyl azodicarboxylic acid to obtain an object substance (I-27).

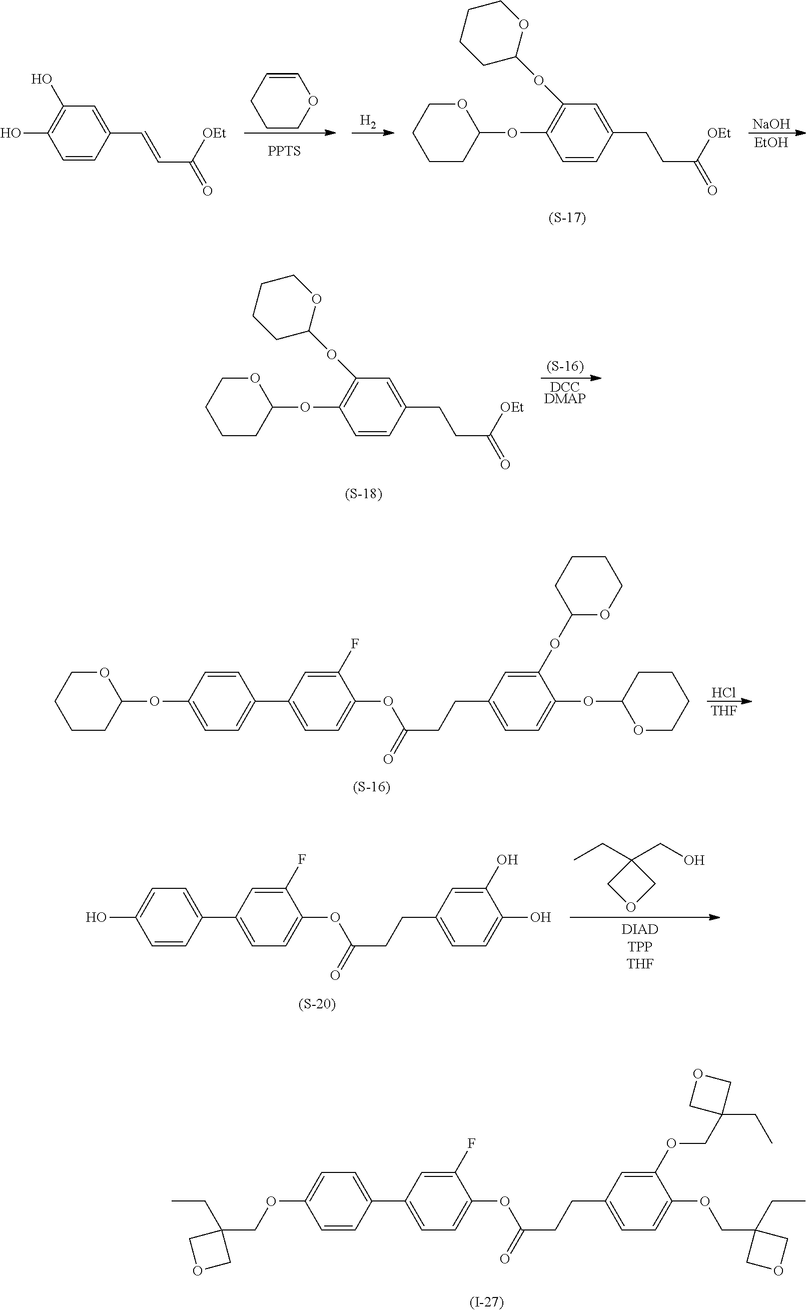

In the present invention, a composition containing a polymerizable compound represented by General Formula (I) as an essential component, and a polymerizable compound represented by General Formula (II) which may be added as necessary is referred to as a polymerizable composition, and further a composition containing the polymerizable compound or a polymerizable composition and one or more liquid crystal compounds is referred to as a liquid crystal composition containing the polymerizable compound. In addition, the polymerizable compound according to the present invention is preferably a liquid crystalline compound.

Other polymerizable compounds may be added the polymerizable composition and the liquid crystal composition containing the polymerizable compound of the present invention within an arbitrary range, in addition to at least one polymerizable compound of the present invention to be used, for the polymerizable composition and the liquid crystal composition containing the polymerizable compound of the present invention. The specific examples of the polymerizable compound other than those of the present invention are not particularly limited. However, the examples of the polymerizable liquid crystal compound to be used in combination preferably include those having an acryloyloxy group or a methacryloyloxy group (R-I) in the compound, and more preferably those having two or more polymerizable functional groups within a molecule.

The specific examples of the polymerizable (liquid crystal) compound to be used in combination include a compound represented by General Formula (II):

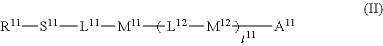

(in the formula, R¹¹ is a polymerizable group, S¹¹ independently represents a single bond, or a C1 to C12 alkylene group, in which, at least one —CH₂— is a group in which a carbon atom may be substituted with an oxygen atom, —COO—, —OCO— or —OCOO—, provided that the oxygen atoms do not directly bond to each other, L¹¹ and L¹² each independently represent a single bond, —O—, —S—, —OCH₂—, —CH₂O—, —CO—, —COO—, —OCO—, —OCOOCH₂—, —CH₂OCOO—, —CO—NR¹³—, —NR¹³—CO—, —CH═N—, —SCH₂—, —CH₂S—, —CH═CH—COO—, —OOC—CH═CH—, —COOC₂H₄—, —OCOC₂H₄—, —C₂H₄OCO—, —C₂H₄COO—, —OCOCH₂—, —CH₂COO—, —CH═CH—, —C₂H₄—, —CF═CH—, —CH═CF—, —CF₂—, —CF₂O—, —OCF₂—, —CF₂CH₂—, —CH₂CF₂—, —CF₂CF₂ or —C≡C— (in the formula, R¹³ represents a C1 to C4 alkyl group), M¹¹ and M¹² each independently represent a 1,4-phenylene group, a 1,4-cyclohexylene group, a pyridine-2,5-diyl group, a pyrimidine-2,5-diyl group, a naphthalene-2,6-diyl group, a tetrahydronaphthalene-2,6-diyl group or a 1,3-dioxane-2,5-diyl group, and M¹ and M¹ each independently are not substituted, or may be substituted with an alkyl group, a halogenated alkyl group, an alkoxy group, a halogenated alkoxy group, a halogen group, a cyano group, or a nitro group, and l¹¹ represents 0, 1, 2, or 3; when l¹¹ represents 2 or 3, L¹² and M¹², each respectively having 2 or 3, may be the same as or different from each other).

With regard to the compound represented by General Formula (II), L¹¹ and L¹² each independently are preferably a single bond, —O—, —COO— or —OCO—, M¹¹ and M¹² each independently are a 1,4-phenylene group, a 1,4-cyclohexylene group, a pyridine-2,5-diyl group, a pyrimidine-2,5-diyl group or a naphthalene-2,6-diyl group.

The examples of the compound represented by General Formula (II) preferably include compounds represented by General Formula (II-1) to General Formula (II-43).

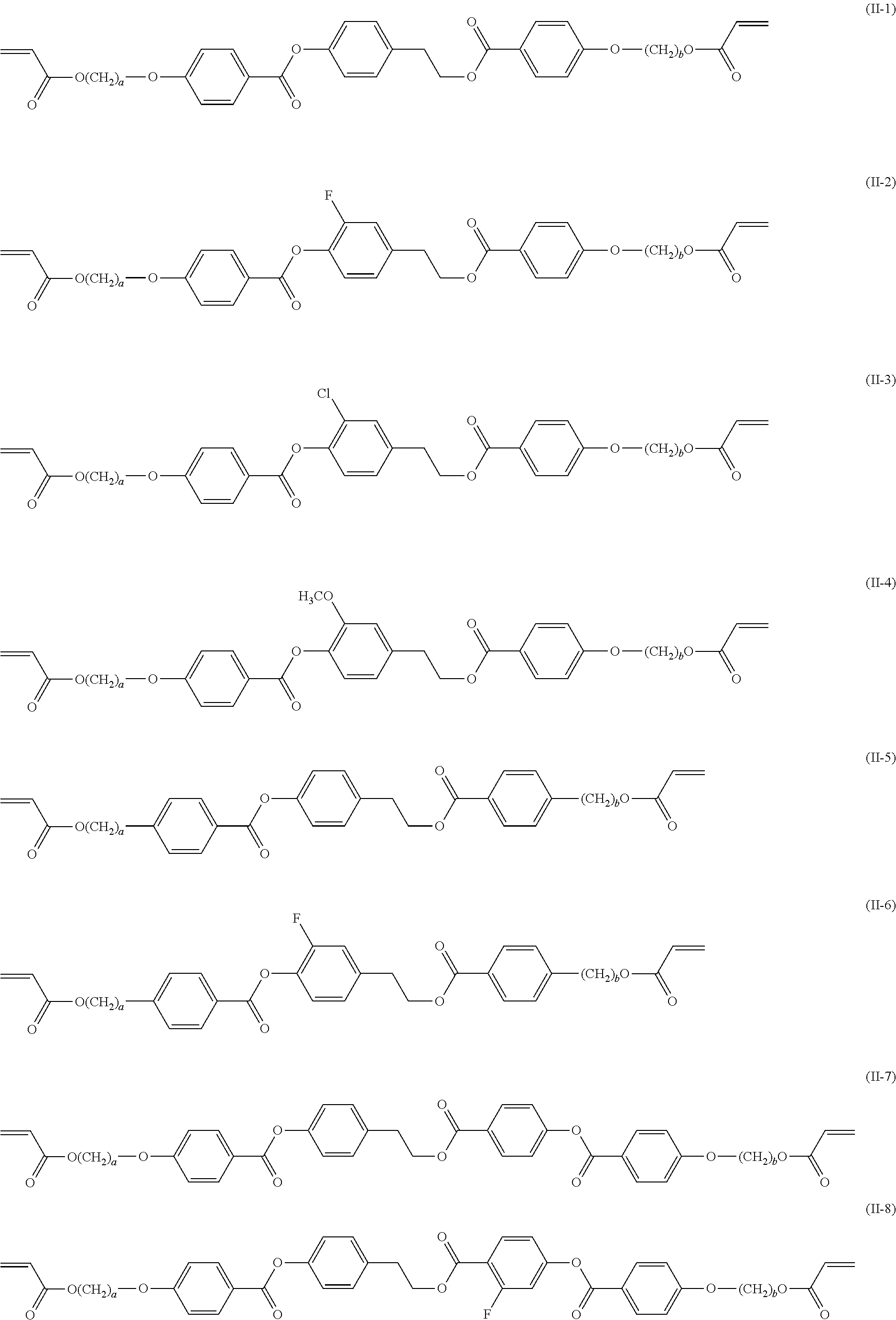

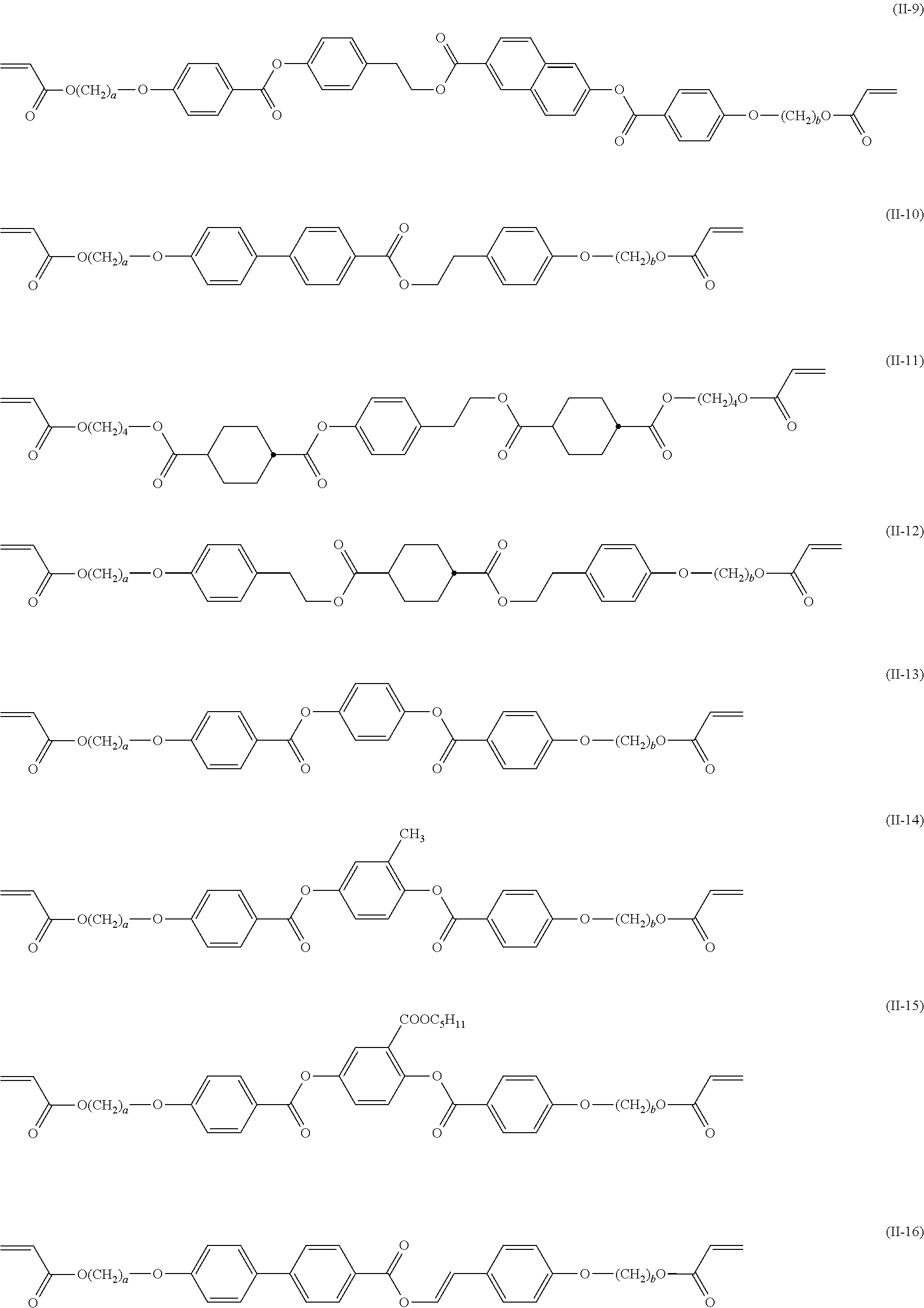

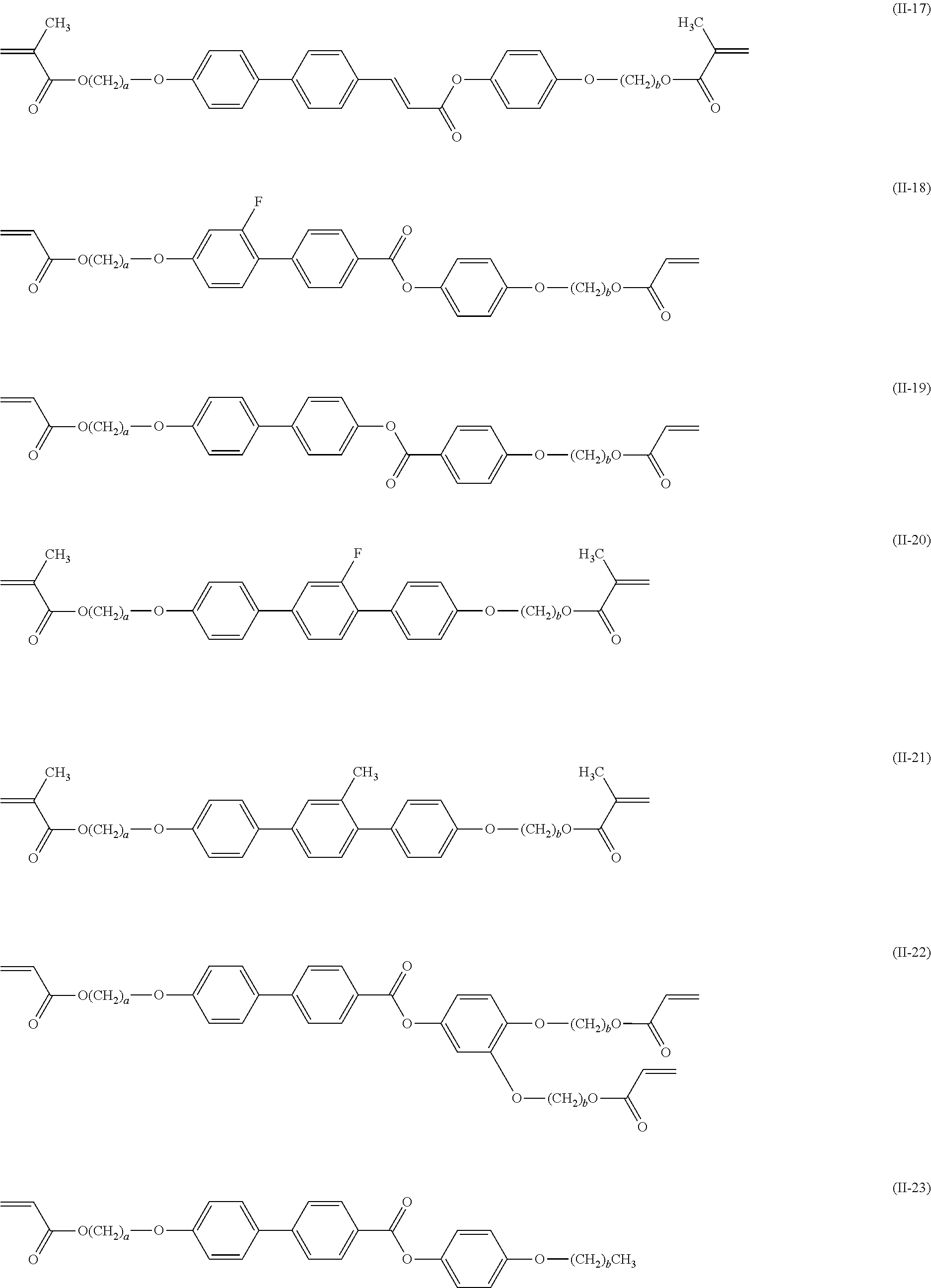

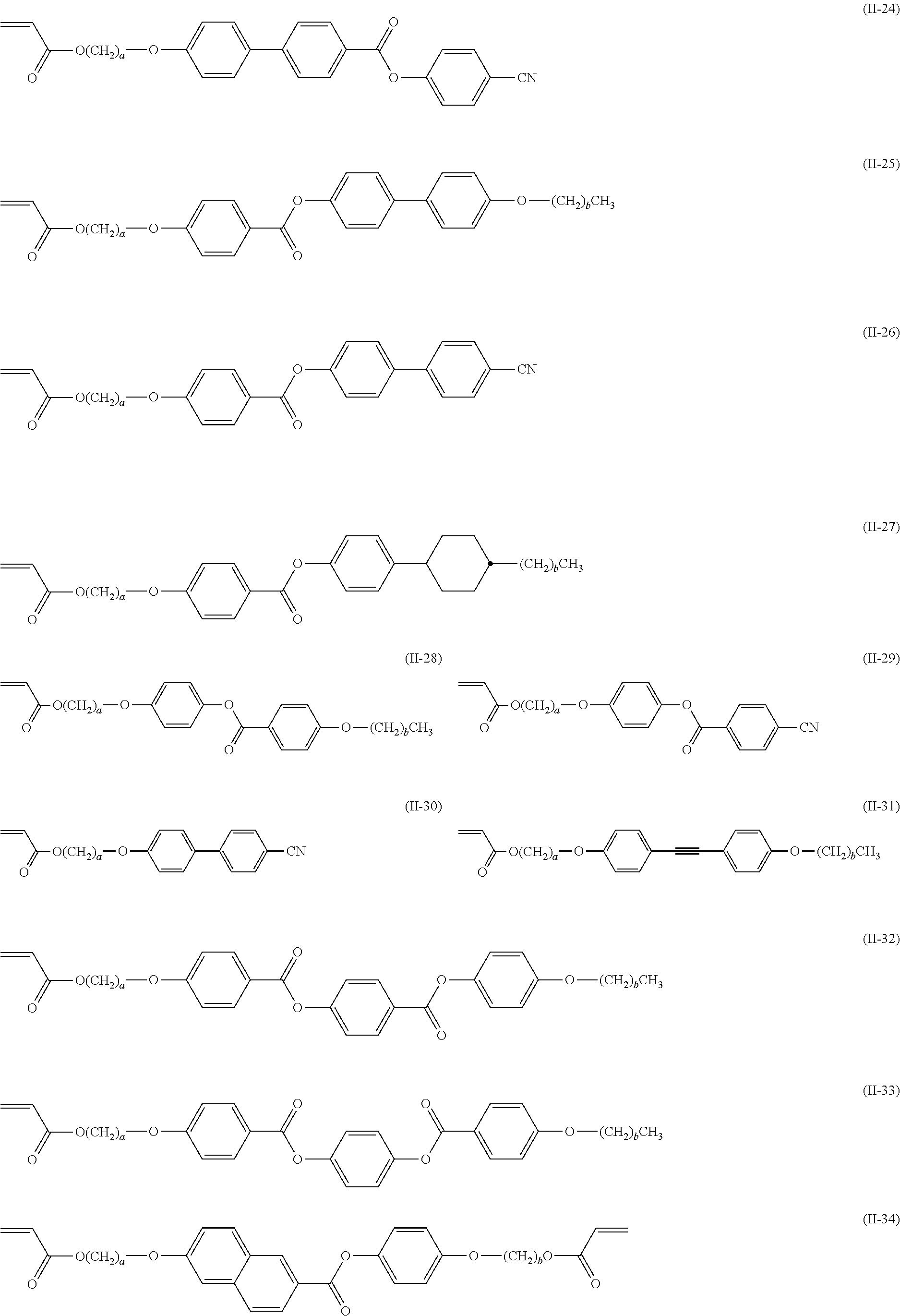

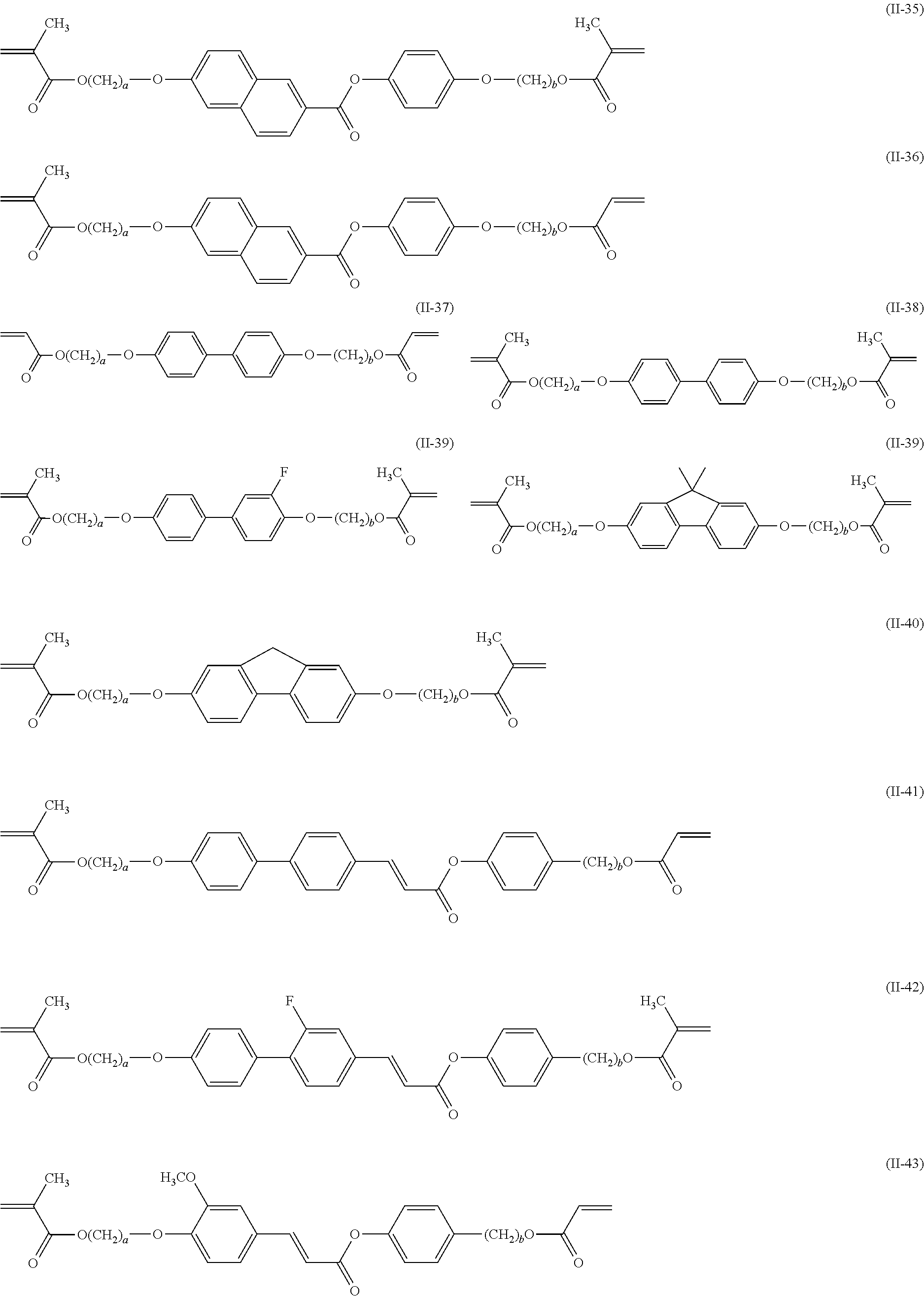

(In the formula, a and b represent an integer of 0 to 12, and when a and/or b is 0 and oxygen atoms directly bond to each other, one of the oxygen atoms is removed.)

The polymerizable compound of the present invention is useful as a constituent component when preparing an optical compensation film used for a polarizing plate, a retardation plate, or the like, and is useful for a PSA (Polymer Sustained Alignment) type liquid crystal display, and a PSVA (Polymer Stabilised Vertical Alignment) type liquid crystal display, in which an alignment of liquid crystal molecules is controlled by the polymerizable compound. In addition, the polymerizable compound can be used for an OCB (Optically Compensated Birefringence) -LCD and an IPS-LCD (In-plane Switching liquid crystal display element). As an example of the driving mode of the liquid crystal display, active driving and passive driving can be used, the compound is useful for an AM-LCD (Active Matrix liquid crystal display element), a TN (Nematic liquid crystal display element) and a STN-LCD (Super-twisted nematic liquid crystal display element), and is particularly useful for an AM-LCD.

As a non-polymerizable liquid crystal composition, a fluorine-based nematic liquid crystal composition having a positive or a negative dielectric anisotropy, a tolan-based nematic liquid crystal composition having a positive or a negative dielectric anisotropy, a cyano-based nematic liquid crystal composition having a positive dielectric anisotropy, a ferroelectric liquid crystal composition, a blue phase liquid crystal composition, a cholesteric liquid crystal composition, or the like, which are generally known, can be used. In a case where the liquid crystal composition of the present invention is a cholesteric liquid crystal composition, commonly, a chiral compound is added. The specific examples of the compound include the compounds represented by General Formula (IV-1) to General Formula (IV-7). A blending amount of the chiral compound is preferably 0.5 weight % to 30 weight %, and more preferably 2 weight % to 20 weight % with respect to the liquid crystal composition.

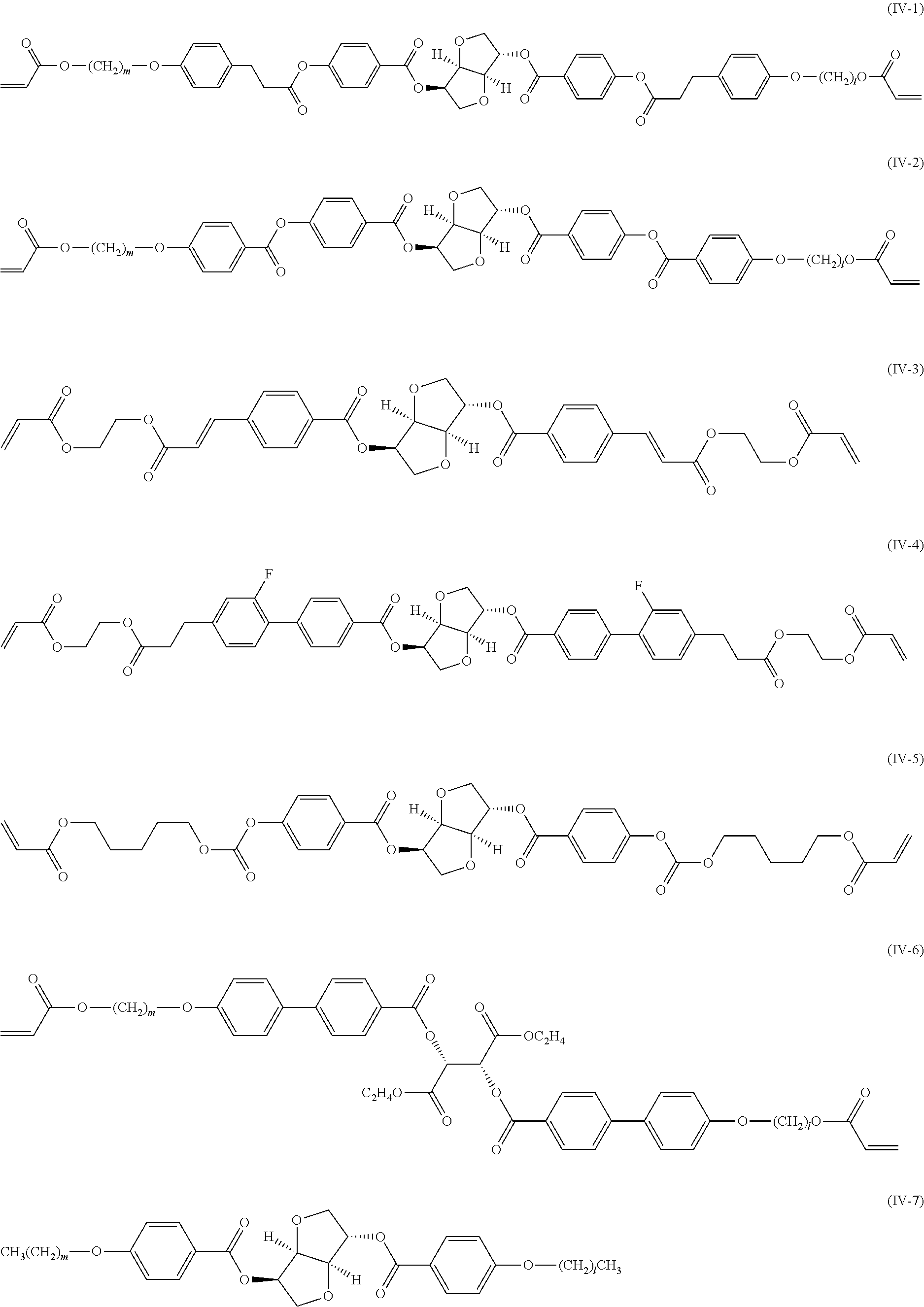

(In the formula, m and l represent an integer of 0 to 12, and when m and/or l is 0 and oxygen atoms directly bond to each other, one of the oxygen atoms is removed.)

In a case of the PSA, PS-VA, PS-IPS and PS-OCB liquid crystal compositions using the polymerizable compound of the present invention, at least one type of the polymerizable compound represented by General Formula (I) is contained, but 1 type to 5 types are preferably contained, and 1 type to 3 types are particularly preferably contained. In addition, the lower limit content of the polymerizable compound represented by General Formula (I) is preferably 0.01 mass % and more preferably 0.03 mass %, and the upper limit content thereof is preferably 5.0 mass % and more preferably 1.0 mass %, since when the content of the polymerizale compound is small, an alignment regulation force with respect to the non-polymerizable liquid crystal compound becomes small, and when the content of the polymerizable compound is too large, the energy necessary for polymerization is increased, and the amount of the polymerizable compound which is not polymerized is increased.

In addition, a compound, which does not exhibit liquid cyrstalline properties, can be added to the polymerizable (liquid crystal) composition of the present invention. If the compound is commonly recognized as a polymer forming monomer or a polymer forming oligomer in this technical field, it can be used without particular limitation. In a case where the polymerizable composition is required to exhibit a liquid cyrstalline phase, an addition amount of the compound needs to be adjusted such that the liquid crystal composition containing the polymerizable compound exhibits liquid crystalline properties after addition.

The polymerizable (liquid crystal) composition of the present invention can be polymerized by heat and light without adding a polymerization initiator, since the composition has biphenyl and phenylnaphthalene skeletons in which π electrons are widely conjugated, but the photopolymerization initiator may be added. The concentration of the photopolymerization initiator to be added is preferably 0.1 mass % to 10 mass %, more preferably 0.2 mass % to 10 mass %, and particularly preferably 0.4 mass % to 5 mass %. The examples of the photoinitiator include benzoin ethers, benzophenones, acetophenones, benzyl ketals, acyl phosphine oxides, or the like.

In addition, a stabilizer can be added to the polymerizable (liquid crystal) composition of the present invention, in order to improve storage stability of the composition. The examples of the stabilizer to be used include, for example, hydroquinones, hydroquinone monoalkyl ethers, tertiary butyl catechols, pyrogallols, thiophenols, nitro compounds, β-naphtylamines, β-naphthols, nitroso compounds, or the like. In a case where the stabilizer is used, an addition amount thereof is preferably in a range of 0.005 mass % to 1 mass %, more preferably 0.02 mass % to 0.5 mass %, and particularly preferably 0.03 mass % to 0.1 mass % with respect to the polymerizable composition.

In addition, in a case where the polymerizable (liquid crystal) composition of the present invention is used for purposes such as a raw material of a retardation film, a polarizing film or an alignment film, a printing ink and a paint, a protection film, or the like, a metal, a metal complex, a dye, a pigment, a solvent, a coloring material, a fluorescent material, a phosphorescent material, a surfactant, a leveling agent, a thixotropic agent, a gelling agent, polysacharrides, an ultraviolet ray absorbing agent, an infrared ray absorbing agent, an antioxidant, an ion exchange resin, metal oxides such as titanium oxide, or the like can be added according to the purpose.

Next, the optically anisotropic material of the present invention is described. The optically anisotropic material which is manufactured by polymerizing the polymerizable (liquid crystal) composition of the present invention can be used for various purposes. For example, in a case where the liquid crystal composition containing the polymerizable compound of the present invention is polymerized in a state where the molecules are not aligned, the composition can be used as a light scattering plate, a depolarizing plate, and a moiré fringe preventing plate. In addition, the optically anisotropic material which is manufactured by polymerizing the liquid crystal composition containing the polymerizable compound of the present invention in a state where molecules are aligned, has optical anisotropy as physical properties, which is useful. The optically anisotropic material of the present invention can be manufactured by for example, polymerizing the liquid crystal of the present invention, after allowing a surface having the liquid crystal composition containing the polymerizable compound carried thereon to be carried on a substrate having undergone the rubbing process with a fabric, a substrate having a surface provided with an organic thin film and having undergone the rubbing process with a fabric, or a substrate having an alignment film with SiO₂ obliquely vapor-deposited, or to be interposed between the substrates.

The examples of the method of allowing the substrate to carry the liquid crystal composition containing the polymerizable compound includes a spin coating, a die coating, an extrusion coating, a roll coating, a wire bar coating, a gravure coating, a spray coating, a dipping, a printing, or the like. In addition, when coating, the liquid crystal composition containing the polymerizable compound may be used as is, or an organic solvent may be added thereto. The examples of the organic solvent include ethyl acetate, tetrahydrofuran, toluene, hexane, methanol, ethanol, dimethyl formamide, dichloromethane, isopropanol, acetone, methylethylketone, acetonitrile, cellosolve, cyclohexanone, γ-butyl lactone, acetoxy-2-ethoxyethane, propylene glycol monomethyl acetate, N-methyl pyrrolidinone, or the like. The above may be used independently or in combination. The above may be appropriately selected in consideration of steam pressure and the solubility of the liquid crystal composition containing the polymerizable compound. In addition, the addition amount thereof is preferably equal to or less than 90 weight %. As a method of volatilizing the added organic solvent, air drying, heated air drying, reduced pressure drying, and reduced pressure heated air drying can be used. In order to further improve the coating properties of the polymerizable liquid crystal material, it is effective to provide an intermediate layer such as a polyimide thin film on the substrate, or add a leveling agent to the polymerizable liquid crystal material. In a case where the adhesion between the substrate and the optically anisotropic material obtained by polymerizing the polymerizable liquid crystal material is poor, it is effective to provide an intermediate layer such as a polyimide thin film on the substrate as means for improving adhesiveness.

The examples of the method of allowing the liquid crystal composition containing the polymerizable compound to be interposed between the substrates include an injection method using a capillary action. Means for injecting a liquid crystal material after lowering pressure of a space formed between the substrates, or one drop fill (ODF) is effective.

The examples of an alignment process other than the rubbing process, or the oblique vapor-deposition of SiO₂ include using a fluid flow alignment of the liquid crystal material, or using an electric field or a magnetic field. The above alignment means may be used independently or used in combination. Further, the examples of the alignment process replacing the rubbing include using a photo-alignment method. According to this method, for example, an organic thin film having a functional group dimerized within a molecule such as polyvinyl cinnamate, an organic thin film having a functional group isomerized by light, or an organic thin film such as polyimide is irradiated with polarized light, and preferably with a polarized ultraviolet ray to form an alignment film. Since alignment patterning is easily achieved by applying a photomask to the photo-alignment method, it is possible to precisely control molecule alignment within the optically anisotropic material.

With regard to a shape of the substrate, other than a flat plate, the substrate may have a curved surface as a constituent portion. As the material configuring the substrate, both an organic material and an inorganic material can be used. The examples of the organic material as the material of the substrate include polyethylene terephthalate, polycarbonate, polyimide, polyamide, methyl polymethacrylate, polystyrene, polyvinyl chloride, polytetrafluoroethylene, polychlorotrifluoroethylene, polyacrylate, polysulfone, triacetyl cellulose, cellulose, polyether ether ketone, or the like. In addition, the examples of the inorganic material include silicon, glass, calcite, or the like.

In a case where appropriate aligning properties cannot be obtained by rubbing the substrate with a fabric, the organic thin film such as a polyimide thin film or a polyvinyl alcohol thin film may be formed on the substrate surface and rubbed with a fabric according to a well known method. In addition, a polyimide thin film imparting a pre-tilt angle commonly used for a TN liquid crystal device or a STN liquid crystal device is particularly preferable, since the structure of the molecule alignment within the optically anisotropic material is more precisely controlled.

In addition, in a case where an alignment state is controlled using an electric field, a substrate having an electrode layer is used. In this case, it is preferable to form the organic thin film such as the above mentioned polyimide thin film on the electrode.

As a method of polymerizing the liquid crystal composition of the present invention, a method of polymerizing by irradiating the composition with an active energy ray such as an ultraviolet ray, an electron beam, or the like is preferable, since rapid polymerization is desirable. In a case of using the ultraviolet ray, a polarized light source may be used, or a non polarized light source may be used. In addition, in a case where the liquid crystal composition is polymerized in a state where the composition is interposed between the two substrates, at least a side of the substrate irradiated with the active energy ray has to be appropriately transparent thereto. In addition, means may be used in which only a specific portion is polymerized using a mask upon irradiation with light, and then an alignment state of the non polymerized portion is changed by changing conditions such as an electric field, a magnetic field, a temperature, or the like, and further the composition is irradiated with the active energy ray to be polymerized. In addition, the temperature upon irradiation is preferably within a temperature range in which a liquid crystal state of the liquid crystal composition of the present invention is maintained. In particular, when manufacturing the optically anisotropic material by photopolymerization, the polymerization is preferably performed at a temperature as close to room temperature as possible, that is, typically at a temperature of 25° C., from a viewpoint of avoiding induction of unintended thermal polymerization. The intensity of the active energy ray is preferably 0.1 mW/cm² to 2 W/cm². When the intensity is equal to or lower than 0.1 mW/cm², a long period of time is necessary for completing photopolymerization, and productivity is degraded. When the intensity is equal to or higher than 2 W/cm², there is a concern that the polymerizable liquid crystal compound or the liquid crystal composition containing the polymerizable compound may be deteriorated.

The optically anisotropic material of the present invention obtained by polymerization can be subjected to a thermal process for the purpose of reducing a change of initial properties and realizing stable properties. The temperature of the thermal process is preferably within a range from 50° C. to 250° C., and the period of time for the thermal process is preferably 30 seconds to 12 hours.

The optically anisotropic material of the present invention manufactured according to this method may be used independently by separating it from the substrate, and may also be used without separating it from the substrate. In addition, the obtained optically anisotropic material may be laminated, or may be adhered to other substrates.

### EXAMPLES

### Example 1

40 g (155 millimole) of 2-(4-bromophenoxy)tetrahydropyran, 21 g (155 millimole) of 4-hydroxyphenyl boric acid, 32 g (232 millimole) of potassium carbonate, 1.8 g of tetrakis triphenylphosphine palladium, 200 ml of tetrahydrofuran, and 100 ml of pure water were put into a reactor vessel equipped with a stirring apparatus, a condenser, and a thermometer, and reacted at a temperature of 70° C. for 5 hours. After the reaction was finished, the resultant was cooled, and 10% hydrochloric acid was added thereto, and then an object substance was extracted using ethyl acetate. An organic layer was washed with water and a saturated saline solution, and a solvent was removed by distillation. After that, the object substance was dispersion-washed using toluene, and refined using alumina column, thereby obtaining 27 g of a compound represented by Formula (1).

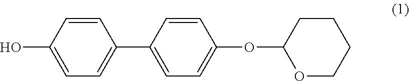 [SMILES: Oc1ccc(-c2ccc(OC3CCCCO3)cc2)cc1]

Next, 15 g (55 millimole) of the compound represented by the Formula (1, 7 g (83 millimole) of a methacrylic acid, 400 mg of dimethylaminopyridine, and 150 ml of dichloromethane were put into a reactor vessel equipped with a stirring apparatus, a condenser, and a thermometer, the reactor vessel was held in an ice-cooled bath at a temperature of equal to or less than 5° C., and 8.3 g (66 millimole) of diisopropylcarbodiimide was slowly added dropwise under the atmosphere of nitrogen gas. After dropwise addition was finished, the reactor vessel was brought back to room temperature and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined using a double amount (weight ratio) of alumina column, dispersion-washed using a mixed solution of dichloromethane and methanol, thereby obtaining 18 g of a compound represented by Formula (2).

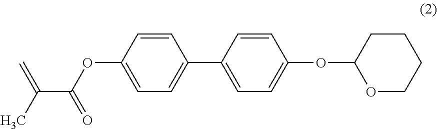 [SMILES: C=C(C)C(=O)Oc1ccc(-c2ccc(OC3CCCCO3)cc2)cc1]

Further, 18 g of the compound represented by the Formula (2 and 100 ml of THF were put into a reactor vessel equipped with a stirring apparatus and a thermometer, a mixed solution of 10 ml of a methanol solution and 1 ml of a hydrochloric acid were slowly added dropwise thereto. After dropwise addition was finished, the resultant was further reacted for 2 hours. After the reaction was finished, 200 ml of ethyl acetate was added to the reaction liquid, an organic layer was washed with pure water, saturated sodium hydrogen carbonate, and a 5% aqueous hydrochloric acid, further washed with a saturated saline solution, and an organic layer was dried with anhydrous sodium sulfate. A solvent was removed by distillation, thereby obtaining 11 g of a compound represented by Formula (3.

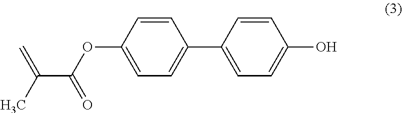 [SMILES: C=C(C)C(=O)Oc1ccc(-c2ccc(O)cc2)cc1]

2 g of the compound represented by Formula (3), 2.3 g of 3-(3,4-acryloyloxy)phenyl)propionic acid, 150 mg of dimethylaminopyridine, and 50 ml of dichloromethane were put into a reactor vessel equipped with a stirring apparatus, a condenser, and a thermometer, the reactor vessel was held in an ice-cooled bath at a temperature of equal to or less than 5° C., and 1.2 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of silica gel, thereby obtaining 2 g of a compound represented by Formula (4).

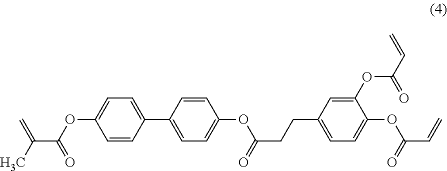 [SMILES: C=CC(=O)Oc1ccc(CCC(=O)Oc2ccc(-c3ccc(OC(=O)C(=C)C)cc3)cc2)cc1OC(=O)C=C]

(Physical Property Value)

¹H-NMR (solvent: deuterochloroform): δ: 2.06 (s, 3H), 2.91 to 2.98 (m, 2H), 3.08 to 3.12 (m, 2H), 5.77 (s, 1H), 5.99 to 6.07 (d, 2H), 6.23 to 6.30 (m, 2H), 6.37 (s, 1H), 6.55 (s, 1H), 6.59 (s, 1H), 7.09 (d, 2H), 7.17 to 7.25 (m, 5H), 7.54 to 7.57 (m, 4H)

¹³C-NMR (solvent: deuterochloroform): δ: 18.4, 30.2, 35.6, 121.8, 121.9, 123.3, 126.7, 127.1, 127.3, 128.1, 133.1, 135.8, 138.0, 138.1, 139.0, 140.5, 149.9, 150.3, 163.4, 171.1

Infrared absorption spectrum (IR)(KBr): 1760, 1652 to 1622, 809 cm⁻¹

Melting point: 117° C.

### Example 2

9 g of 2-((6-bromonaphthalene-2-yl)oxy)tetrahydro 2H pyran, 4.5 g (32 millimole) of a hydroxyphenyl boric acid, 6.4 g (46 millimole) of potassium carbonate, 400 mg of tetrakis triphenylphosphine palladium, 200 ml of tetrahydrofuran, and 100 ml of pure water were put into a reactor vessel equipped with a stirring apparatus, a condenser, and a thermometer, and reacted at a temperature of 70° C. for 5 hours. After the reaction was finished, the resultant was cooled, and a 10% hydrochloric acid was added thereto, and then an object substance was extracted using ethyl acetate. An organic layer was washed with water and a saturated saline solution, and a solvent was removed by distillation. After that, the object substance was dispersion-washed using toluene, thereby obtaining 7 g of a compound represented by Formula (5).

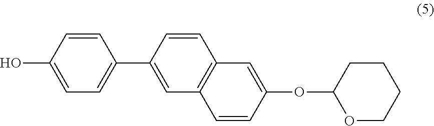 [SMILES: Oc1ccc(-c2ccc3cc(OC4CCCCO4)ccc3c2)cc1]

Next, 7 g of the compound represented by the Formula (5), 2.8 g of a methacrylic acid, 160 mg of dimethylaminopyridine, and 50 ml of dichloromethane were put into a reactor vessel equipped with a stirring apparatus, a condenser, and a thermometer, the reaction vessel was held in an ice-cooled bath at a temperature of equal to or less than 5° C., and 3.3 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of activated alumina, thereby obtaining 9 g of a compound represented by Formula (6).

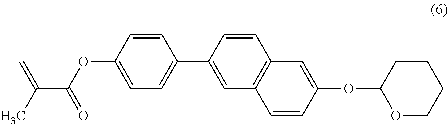 [SMILES: C=C(C)C(=O)Oc1ccc(-c2ccc3cc(OC4CCCCO4)ccc3c2)cc1]

Further, 9 g of the compound represented by the Formula (6) and 100 ml of THF were put into a reactor vessel equipped with a stirring apparatus and a thermometer, a mixed solution of 10 ml of a methanol solution and 1 ml of a hydrochloric acid were slowly added dropwise thereto. After dropwise addition was finished, the resultant was further reacted for 2 hours. After the reaction was finished, 200 ml of ethyl acetate was added to the reaction liquid, an organic layer was washed with pure water, saturated sodium hydrogen carbonate, and a 5% aqueous hydrochloric acid, further washed with a saturated saline solution, and an organic layer was dried with anhydrous sodium sulfate. A solvent was removed by distillation, the resultant is recrystallized with toluene, thereby obtaining 6 g of a compound represented by Formula (7).

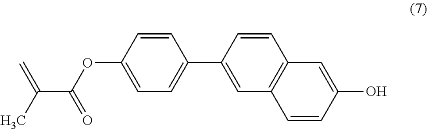 [SMILES: C=C(C)C(=O)Oc1ccc(-c2ccc3cc(O)ccc3c2)cc1]

4 g of the compound represented by the formula (7), 3.7 g of 3-(3,4-acryloyloxy)phenyl)propionic acid, 150 mg of dimethylaminopyridine, and 50 ml of dichloromethane were put into a reactor vessel equipped with a stirring apparatus, a condenser, and a thermometer, the reactor vessel was held in an ice-cooled bath at a temperature of equal to or less than 5° C., and 2 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of silica gel, thereby obtaining 4.2 g of a compound represented by Formula (8).

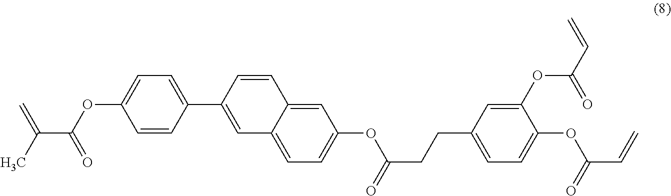 [SMILES: C=CC(=O)Oc1ccc(CCC(=O)Oc2ccc3cc(-c4ccc(OC(=O)C(=C)C)cc4)ccc3c2)cc1OC(=O)C=C]

(Physical Property Value)

¹H-NMR (solvent: deuterochloroform): δ: 2.09 (s, 3H), 2.94 to 2.98 (m, 2H), 3.11 to 3.14 (m, 2H), 5.78 (s, 1H), 5.99 to 6.07 (d, 2H), 6.24 to 6.31 (m, 2H), 6.38 (s, 1H), 6.56 (s, 1H), 6.60 (s, 1H), 7.18 to 7.25 (m, 6H), 7.52 to 7.53 (m, 1H), 7.70 to 7.74 (m, 3H), 7.86 to 7.94 (m, 2H), 8.06 (s, 1H),

¹³C-NMR (solvent: deuterochloroform): δ: 18.3, 30.2, 35.6, 118.3, 121.6, 121.9, 123.3, 123.4, 125.5, 126.7, 127.1, 127.3, 128.2, 129.6, 132.8, 133.1, 135.8, 137.6, 138.4, 139.0, 140.5, 141.9, 148.3, 150.4, 163.4, 165.8, 171.1

Infrared absorption spectrum (IR)(KBr): 1760, 1652 to 1622, 809 cm⁻¹

Melting point: 140° C.

### Example 3

8 g of 3′-fluoro-4′-hydroxy[1,1′-biphenyl]-4-yl methacrylate, 17 g of a 3-(3,4-bis(tetrahydro 2H pyran 2-yl)oxy)phenyl)propionic acid, 150 mg of dimethylaminopyridine, and 100 ml of dichloromethane were put into a reactor vessel equipped with a stirring apparatus, a condenser, and a thermometer, the reactor vessel was held in an ice-cooled bath at a temperature of equal to or less than 5° C., and 4.4 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of activated alumina, thereby obtaining 20 g of a compound represented by Formula (9).

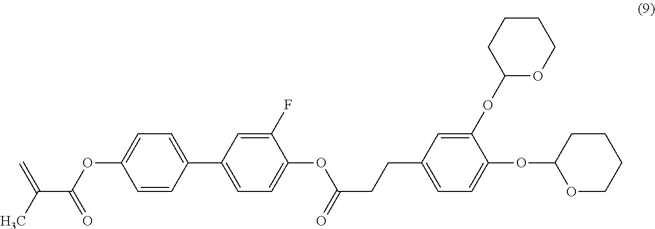 [SMILES: C=C(C)C(=O)Oc1ccc(-c2ccc(OC(=O)CCc3ccc(OC4CCCCO4)c(OC4CCCCO4)c3)c(F)c2)cc1]

Further, 4 g of the compound represented by the Formula (9) and 100 ml of THF were put into a reactor vessel equipped with a stirring apparatus and a thermometer, a mixed solution of 10 ml of a methanol solution and 1 ml of a hydrochloric acid were slowly added dropwise thereto. After dropwise addition was finished, the resultant was further reacted for 2 hours. After the reaction was finished, 200 ml of ethyl acetate was added to the reaction liquid, an organic layer was washed with pure water, saturated sodium hydrogen carbonate, and a 5% aqueous hydrochloric acid, further washed with a saturated saline solution, and an organic layer was dried with anhydrous sodium sulfate. A solvent was removed by distillation, thereby obtaining 12 g of a compound represented by Formula (10).

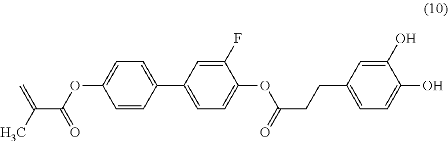 [SMILES: C=C(C)C(=O)Oc1ccc(-c2ccc(OC(=O)CCc3ccc(O)c(O)c3)c(F)c2)cc1]

4 g the compound represented by the Formula (10, 3 g of triethylamine, and 50 ml of tetrahydrofuran were put into a reactor vessel equipped with a stirring apparatus, a condenser, and a thermometer, and the reactor vessel was held in an ice-cooled bath at a temperature of equal to or less than 5° C., and 2.2 g of acrylic acid chloride was slowly added dropwise under the atmosphere of nitrogen gas. After dropwise addition was finished, the reactor vessel was brought back to room temperature, and reacted for 5 hours. After the reaction liquid was filtrated, ethyl acetate was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column using a double amount (weight ratio) of silica gel, thereby obtaining 3 g of a compound represented by Formula (11).

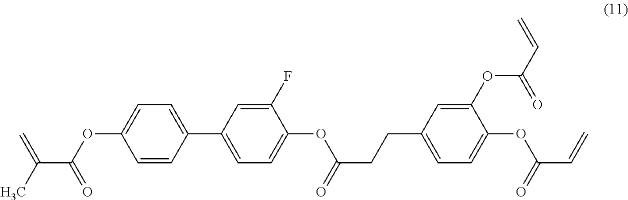 [SMILES: C=CC(=O)Oc1ccc(CCC(=O)Oc2ccc(-c3ccc(OC(=O)C(=C)C)cc3)cc2F)cc1OC(=O)C=C]

(Physical Property Value)

¹H-NMR (solvent: deuterochloroform): δ: 2.08 (s, 3H), 2.95 to 2.98 (m, 2H), 3.10 to 3.13 (m, 2H), 5.78 (s, 1H), 5.98 to 6.01 (d, 2H), 6.21 to 6.30 (m, 2H), 6.37 (s, 1H), 6.55 (s, 1H), 6.59 (s, 1H), 7.11 to 7.15 (t, 2H), 7.18 to 7.25 (m, 4H), 7.31 to 7.37 (m, 2H), 7.54 (d, 2H)

¹³C-NMR (solvent: deuterochloroform): δ: 18.3, 30.1, 35.1, 115.2, 115.3, 122.0, 123.0, 123.3, 123.4, 123.9, 126.6, 127.1, 127.4, 128.0, 133.1, 135.7, 138.8, 140.5, 141.9, 150.7, 163.3, 163.4, 165.7, 170.1

Infrared absorption spectrum (IR)(KBr): 1760, 1652 to 1622, 809 cm⁻¹

Melting point: 91° C.

### Example 4

5.2 g of a 4′-(3-acryloyl)oxypropoxy)-[1,1′-biphenyl]-4-carboxylic acid, 4.2 g of 2-(3,4-acryloyloxy)phenyl)ethanol, 150 mg of dimethylaminopyridine, and 100 ml of dichloromethane were put into a reactor vessel equipped with a stirring apparatus, a condenser, and a thermometer, the reactor vessel was held in an ice-cooled bath at a temperature of equal to or less than 5° C., and 2.5 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the filtrate was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of silica gel, thereby obtaining 6 g of a compound represented by Formula (12).

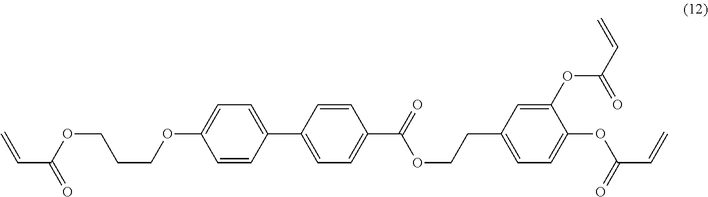 [SMILES: C=CC(=O)OCCCOc1ccc(-c2ccc(C(=O)OCCc3ccc(OC(=O)C=C)c(OC(=O)C=C)c3)cc2)cc1]

(Physical Property Value)

¹H-NMR (solvent: deuterochloroform): δ: 2.11 (m, 2H), 2.93 (t, 2H), 4.20 to 4.29 (m, 4H), 4.51 to 4.58 (m, 2H), 5.78 to 5.85 (m, 3H), 6.08 to 6.15 (m, 3H), 6.37 to 6.42 (m, 3H), 7.05 to 7.10 (m, 3H), 7.21 (d, 1H), 7.34 (s. 1H), 7.68 to 7.72 (d, 2H), 7.75 (d. 2H), 7.91 to 7.96 (m, 2H)

¹³C-NMR (solvent: deuterochloroform): δ: 28.4, 34.2, 64.8, 65.2, 66.4, 66.5, 114.7, 119.4, 126.0, 127.3, 128.1, 128.4, 129.2, 130.3, 130.7, 133.6, 134.1, 156.7, 157.3, 164.3

Infrared absorption spectrum (IR)(KBr): 1760, 1652 to 1622, 809 cm⁻¹

Melting point: 180° C.

### Example 5

10 g of 4-bromo-3-fluorophenol, 12 g of a 4-(tetrahydro-2H-pyran-2-yloxy)phenyl boric acid, 11 g of potassium carbonate, 1 g of tetrakis triphenylphosphine palladium, 200 ml of tetrahydrofuran, and 100 ml of pure water were put into a reactor vessel equipped with a stirring apparatus, a condenser, and a thermometer, and reacted at a temperature of 70° C. for 5 hours. After the reaction was finished, the resultant was cooled, and saturated ammonium chloride solution was added thereto, and then an object substance was extracted using ethyl acetate. An organic layer was washed with water and a saturated saline solution, and a solvent was removed by distillation. After that, the object substance was dispersion-washed using toluene, thereby obtaining 12 g of a compound represented by Formula (13).

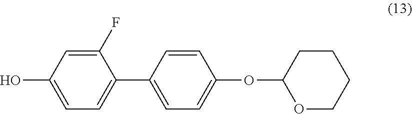 [SMILES: Oc1ccc(-c2ccc(OC3CCCCO3)cc2)c(F)c1]

Next, 6 g of the compound represented by the Formula (13), 2.3 g of a methacrylic acid, 160 mg of dimethylaminopyridine, and 50 ml of dichloromethane were put into a reactor vessel equipped with a stirring apparatus, a condenser, and a thermometer, and the reactor vessel was held in an ice-cooled bath at a temperature of equal to or less than 5° C., and 3.3 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of activated alumina, thereby obtaining 6.5 g of a compound represented by Formula (14).

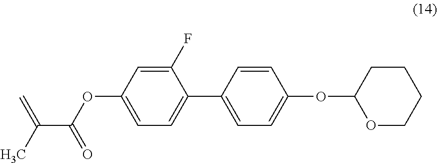 [SMILES: C=C(C)C(=O)Oc1ccc(-c2ccc(OC3CCCCO3)cc2)c(F)c1]

Further, 6.5 g of the compound represented by the Formula (14), and 100 ml of THF were put into a reactor vessel equipped with a stirring apparatus and a thermometer, a mixed solution of 10 ml of a methanol solution and 1 ml of a hydrochloric acid were slowly added dropwise thereto. After dropwise addition was finished, the resultant was further reacted for 2 hours. After the reaction was finished, 200 ml of ethyl acetate was added to the reaction liquid, an organic layer was washed with pure water, saturated sodium hydrogen carbonate, and a 5% aqueous hydrochloric acid, further washed with a saturated saline solution, and an organic layer was dried with anhydrous sodium sulfate. A solvent was removed by distillation, the resultant is recrystallized with toluene, thereby obtaining 3.7 g of a compound represented by Formula (15).

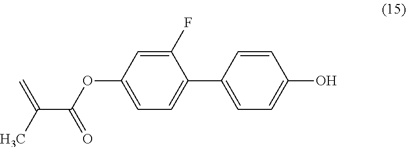 [SMILES: C=C(C)C(=O)Oc1ccc(-c2ccc(O)cc2)c(F)c1]

2.5 g of the compound represented by the Formula (15), 2.7 g of a 3-(3,4-diacryloyloxy)phenyl)propionic acid, 150 mg of dimethylaminopyridine, 50 ml of dichloromethane were put into a reactor vessel equipped with a stirring apparatus, a condenser, and a thermometer, the reactor vessel was held in an ice-cooled bath at a temperature of equal to or less than 5° C., and 2 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% of aqueous hydrochloric acid, further washed with a saturated saline solution, and an organic layer was dried with anhydrous sodium sulfate. After a solvent was removed by distillation, the resultant was refined by column chromatography using a double amount (weight ratio) of silica gel, thereby obtaining 3.5 g of a compound represented by Formula (16).

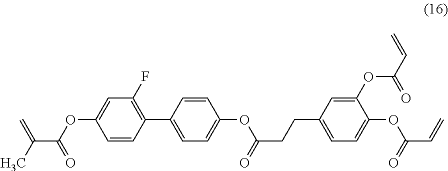 [SMILES: C=CC(=O)Oc1ccc(CCC(=O)Oc2ccc(-c3ccc(OC(=O)C(=C)C)cc3F)cc2)cc1OC(=O)C=C]

(Physical Property Value)

¹H-NMR (solvent: deuterochloroform): δ: 2.08 (s, 3H) 2.92 to 2.95 (m, 2H), 3.09 to 3.12 (m, 2H), 5.80 (s, 1H), 5.99 to 6.03 (d, 2H), 6.22 to 6.38 (m, 2H), 6.38 (s, 1H), 6.56 (s, 1H), 6.61 (s, 1H), 6.98 to 7.02 (m, 2H), 7.08 to 7.12 (m, 2H), 7.13 (s, 1H), 7.21 (s, 2H), 7.31 to 7.41 (m, 1H), 7.45 to 7.64 (m, 2H)

¹³C-NMR (solvent: deuterochloroform): δ: 18.3, 30.1, 35.6, 110.1, 110.3, 117.7, 123.3, 125.7, 126.7, 127.0, 130.0, 132.7, 133.2, 135.4, 139.0, 140.4, 141.8, 150.0, 158.1, 160.6, 163.4, 165, 4, 171.1

Infrared absorption spectrum (IR)(KBr): 1760, 1652 to 1622, 809 cm⁻¹

Melting point: 74.5° C.

### Example 6

25 g of a meldrum's acid, 13 g of t-butanol, and 50 ml of toluene were added to a reactor vessel equipped with a stirring apparatus, a condenser, and a thermometer, heated to a temperature of 100° C., and reacted for 3 hours. After that, the reaction liquid was cooled to a temperature of 60° C., 10 g of 3,4-dihydroxybenzaldehyde and 14 g of pyridine were added thereto, and the liquid was reacted at a temperature of 70° C. for 6 hours. Subsequently, the reaction liquid was washed with a saturated sodium hydrogen carbonate, a 1 N hydrochloric acid, and pure water, an organic layer was dried with anhydrous sodium sulfate. A solvent was removed by distillation, and the resultant was recrystallized with toluene, thereby obtaining 12 g of caffeic acid t-butyl ester represented by Formula (17).

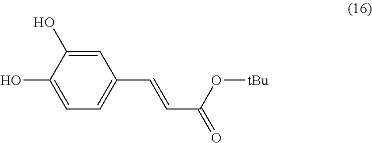 [SMILES: CC(C)(C)OC(=O)/C=C/c1ccc(O)c(O)c1]

Next, 12 g of caffeic acid t-butylester, 600 mg of 5% palladium carbon, and 60 ml of THF were put into an autoclave, and a reduction reaction (room temperature, 8 hours) was performed with hydrogen of 0.3 MPa. After the reaction liquid was filtrated, a reaction solvent was removed by distillation, thereby obtaining 12 g of 3,4-dihydroxyphenylpropionic acid t-butyl represented by Formula (17).

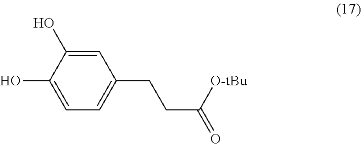

Further, 12 g of a 3,4-dihydroxyphenylpropionic acid t-butyl, 9 g of a methacrylic acid, 140 mg of dimethylaminopyridine, and 100 ml of dichloromethane were put into a reactor vessel equipped with a stirring apparatus, a condenser, and a thermometer, the reactor vessel was held in an ice-cooled bath at a temperature of equal to or less than 5° C., and 14 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of activated alumina, thereby obtaining 15.5 g of a 3,4-dimethacryloyloxyphenylpropionic acid t-butyl compound represented by Formula (18).

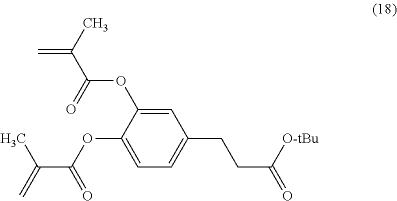

Next, 15.5 g of 3,4-dimethacryloyloxyphenylpropionic acid t-butyl, 50 ml of a formic acid, and 50 ml dichloromethane were added to a reactor vessel equipped with a stirring apparatus and a thermometer, and stirred at room temperature for 3 hours. After the reaction was finished, the reaction liquid was washed with pure water three times, an organic layer was dried with anhydrous sodium sulfate, and a solvent was removed by distillation, thereby obtaining 12 g of 3,4-dimethacryloyloxyphenylpropionic acid represented by Formula (19).

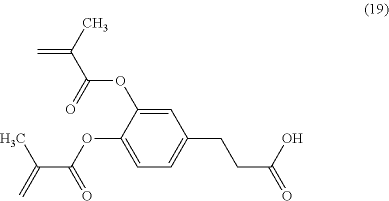 [SMILES: C=C(C)C(=O)Oc1ccc(CCC(=O)O)cc1OC(=O)C(=C)C]

10 g of 3-(3,4-dimethacryloyloxy)phenyl)propionic acid represented by the Formula (19), 8.6 g of 3-fluoro-4-(4′-acryloyloxy-3′-fluoro)phenylphenol, 180 mg of dimethylaminopyridine, and 50 ml of dichloromethane were put into a reactor vessel equipped with a stirring apparatus, a condenser, and a thermometer, the reactor vessel was held in an ice-cooled bath at a temperature of equal to or less than 5° C., and 4.7 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of silica gel, thereby obtaining 12 g of an object compound represented by Formula (20).

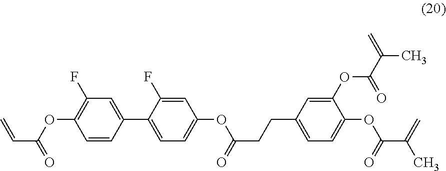 [SMILES: C=CC(=O)Oc1ccc(-c2ccc(OC(=O)CCc3ccc(OC(=O)C(=C)C)c(OC(=O)C(=C)C)c3)cc2F)cc1F]

(Physical Property Value)

¹H-NMR (solvent: deuterochloroform) δ: 2.08 (s, 3H), 2.92 to 2.95 (m, 2H), 3.09 to 3.12 (m, 2H), 5.73 (s, 2H), 6.04 to 6.07 (d, 1H), 6.29 (s, 2H), 6.32 to 6.39 (m, 1H), 6.59 (s, 1H), 6.62 (s, 1H), 6.91 to 6.96 (m, 2H), 7.16 to 7.22 (m, 3H), 7.23 to 7.47 (m, 4H)

¹³C-NMR (solvent: deuterochloroform): δ: 18.3, 30.1, 35.6, 110.1, 110.4, 117.8, 123.4, 124.9, 126.7, 127.0, 128.2, 130.7, 133.2, 134.9, 138.8, 140.5, 141.9, 163.4, 163.5, 170.7

Infrared absorption spectrum (IR)(KBr): 1760, 1652 to 1622, 809 cm⁻¹

Melting point: 87.5° C.

### Example 7

5 g of 4-(4-bromo-2-fluorophenyl)phenol, 4.6 g of a 4-(tetrahydro-2H-pyran-2-yloxy)phenyl boric acid, 3.9 g of potassium carbonate, 0.5 g of tetrakis triphenylphosphine palladium, 100 ml of tetrahydrofuran, and 20 ml of pure water were put into a reactor vessel equipped with a stirring apparatus, a condenser, and a thermometer, and reacted at a temperature of 70° C. for 5 hours. After the reaction was finished, the reaction liquid was cooled, and a saturated ammonium chloride solution was added thereto, and then an object substance was extracted using ethyl acetate. An organic layer was washed with water and a saturated saline solution, and a solvent was removed by distillation. After that, the object substance was dispersion-washed using toluene, thereby obtaining 6.8 g of a compound represented by Formula (21).

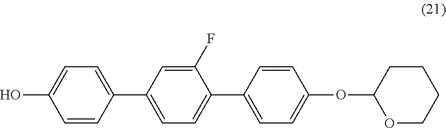 [SMILES: Oc1ccc(-c2ccc(-c3ccc(OC4CCCCO4)cc3)c(F)c2)cc1]

Next, 6.8 g of the compound represented by the Formula (21), 2 g of a methacrylic acid, 150 mg of dimethylaminopyridine, and 50 ml of dichloromethane were put into a reactor vessel equipped with a stirring apparatus, a condenser, and a thermometer, the reactor vessel was held in an ice-cooled bath at a temperature of equal to or less than 5° C., and 2.8 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of activated alumina, thereby obtaining 10.5 g of a compound represented by Formula (22).

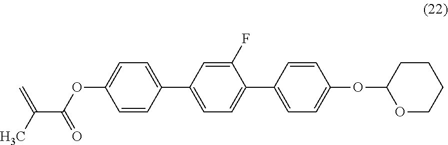 [SMILES: C=C(C)C(=O)Oc1ccc(-c2ccc(-c3ccc(OC4CCCCO4)cc3)c(F)c2)cc1]

Further, 6.5 g of the compound represented by the Formula (22), and 100 ml of THF were put into a reactor vessel equipped with a stirring apparatus and a thermometer, a mixed solution of 10 ml of a methanol solution and 1 ml of a hydrochloric acid were slowly added dropwise thereto. After dropwise addition was finished, the resultant was further reacted for 2 hours. After the reaction was finished, 200 ml of ethyl acetate was added to the reaction liquid, an organic layer was washed with pure water, saturated sodium hydrogen carbonate, and a 5% aqueous hydrochloric acid, further washed with a saturated saline solution, and an organic layer was dried with anhydrous sodium sulfate. A solvent was removed by distillation, and the resultant was recrystallized with toluene, thereby obtaining 4.5 g of a compound represented by Formula (23).

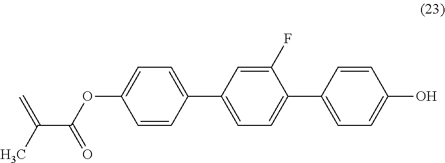 [SMILES: C=C(C)C(=O)Oc1ccc(-c2ccc(-c3ccc(O)cc3)c(F)c2)cc1]

4 g of the compound represented by the Formula (23), 3.5 g of a 3-(3,4-diacryloyloxy)phenyl)propionic acid, 150 mg of dimethylaminopyridine, and 50 ml of dichloromethane were put into a reactor vessel equipped with a stirring apparatus, a condenser, and a thermometer, the reactor vessel was held in an ice-cooled bath at a temperature of equal to or less than 5° C., and 2 g of diisopropylcarbodiimide was slowly added dropwise under the atmosphere of nitrogen gas. After dropwise addition was finished, the reactor vessel was brought back to room temperature, and reacted for 5 hours. After the reaction liquid was filtrated, 150 ml of dichloromethane was added to the filtrate, the obtained mixture was washed with a 5% aqueous hydrochloric acid, further washed with a saturated saline solution, and an organic layer was dried with anhydrous sodium sulfate. After a solvent was removed by distillation, the resultant was refined by column chromatography using a double amount (weight ratio) of silica gel, thereby obtaining 4.8 g of an object compound represented by Formula (24).

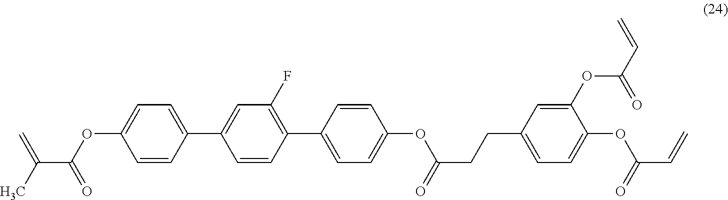 [SMILES: C=CC(=O)Oc1ccc(CCC(=O)Oc2ccc(-c3ccc(-c4ccc(OC(=O)C(=C)C)cc4)cc3F)cc2)cc1OC(=O)C=C]

(Physical Property Value)

¹H-NMR (solvent: deuterochloroform): δ: 2.09 (s, 3H), 2.93 to 2.96 (m, 2H), 3.09 to 3.13 (m, 2H), 5.79 (s, 1H), 6.00 to 6.03 (d, 2H), 6.24 to 6.31 (m, 2H), 6.39 (s, 1H), 6.57 (s, 1H), 6.61 (s, 1H), 7.12 to 7.14 (m, 2H), 7.19 to 7.26 (m, 6H), 7.35 to 7.43 (m, 2H), 7.43 to 7.50 (m, H), 7.52 to 7.63 (m, 3H)

¹³C-NMR (solvent: deuterochloroform): δ: 18.4, 30.2, 35.6, 114.7, 121.6, 122.0, 122.9, 126.7, 127.0, 127.5, 128.0, 129.9, 133.2, 135.8, 138.9, 141.9, 148.3, 150.4, 163.4, 165.8, 171.1

Infrared absorption spectrum (IR)(KBr): 1760, 1652 to 1622, 809 cm⁻¹

Melting point: 128° C.

Liquid cyrstalline phase crystal→128° C. nematic liquid cyrstalline phase→179° C. Isotropic phase

### Example 8

A polymerizable liquid crystal composition (composition 1) having the following composition was prepared.

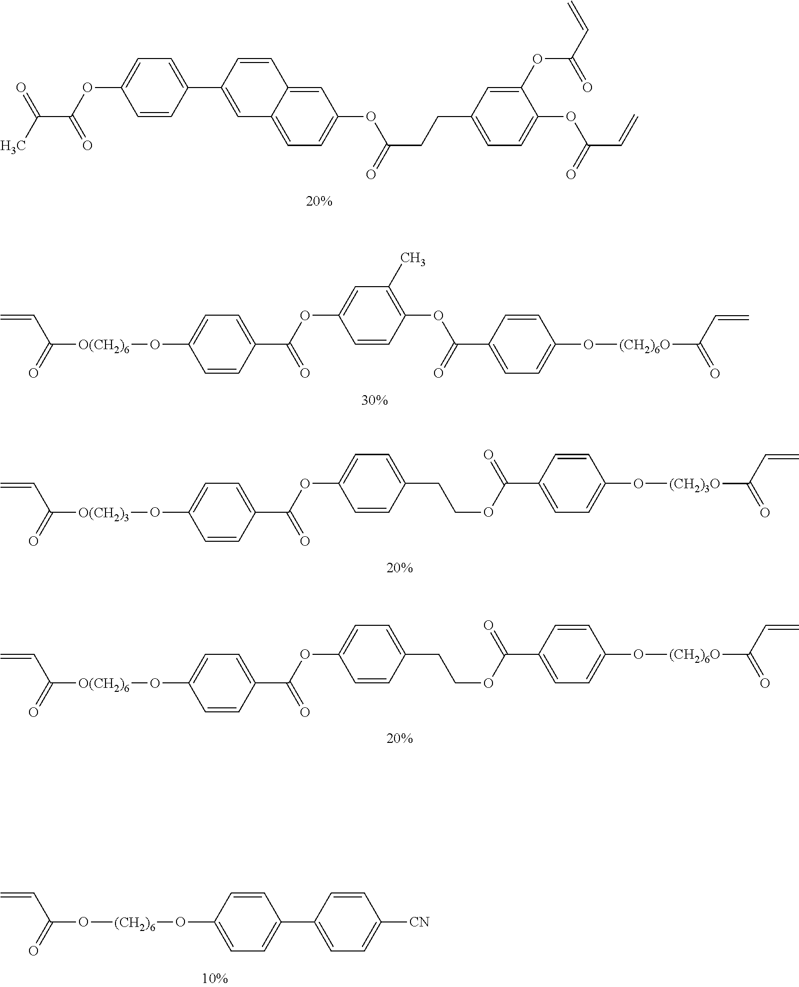

The polymerizable liquid crystal composition has preferable storage stability, and shows a nematic liquid cyrstalline phase in a wide temperature range. 3% of a photopolymerization initiator Irgacure 907 (manufactured by Ciba Speciality Chemicals Co., Ltd.) was added to the polymerizable liquid crystal composition to prepare a polymerizable liquid crystal composition (composition 2). A cyclohexanone solution of the composition 2 was applied on glass having polyimide that had undergone a rubbing process by spin coating, was dried at a temperature of 100° C. for 5 minutes, and then allowed to cool at room temperature, and irradiated with 4 mW/cm² of an ultraviolet ray using a high pressure mercury lamp for 120 seconds. The composition 2 was polymerized while maintaining a state where a molecule is uniformly aligned, thereby obtaining an optically anisotropic material. A surface hardness (by JIS-S-K-5400) of the optically anisotropic material was H. If a phase difference of the obtained optically anisotropic material before heating is 100%, when the optically anisotropic material was heated at a temperature of 240° C. for 1 hour, a phase difference was 92%, and a decreasing ratio of the phase difference was 8%.

### Comparative Example 1

A polymerizable liquid crystal composition (composition 3) having the following composition was prepared.

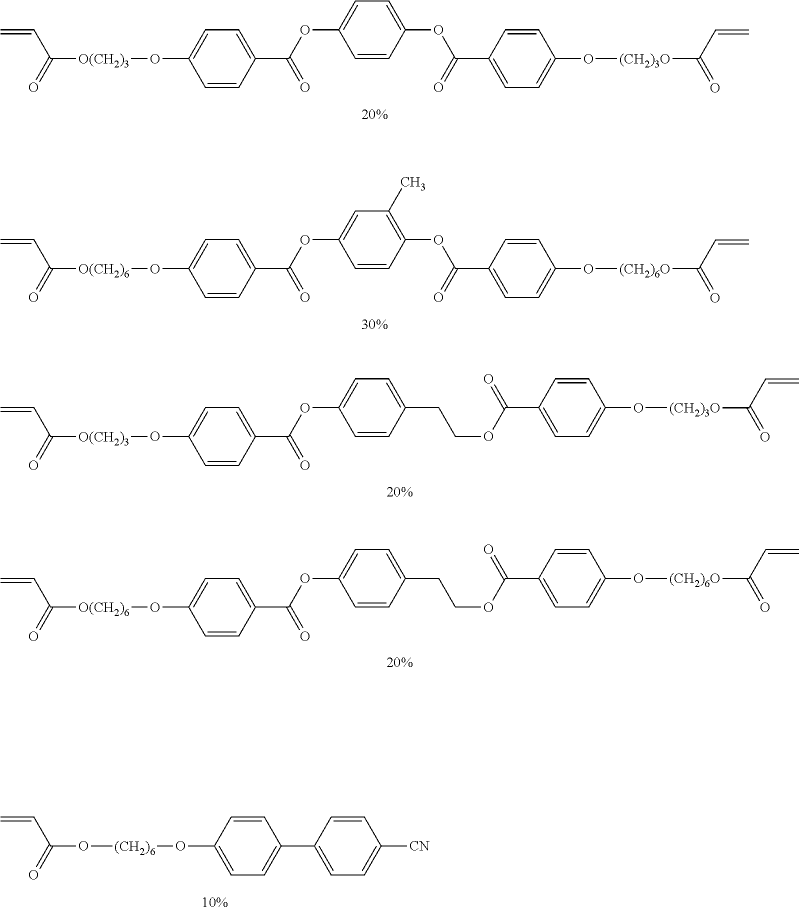

A polymerizable liquid crystal composition shows a nematic liquid crystalline phase, but the storage stability is poor, and thus crystal was precipitated at room temperature in 8 hours.

### Comparative Example 2

A polymerizable liquid crystal composition (composition 4) having the following composition was prepared.

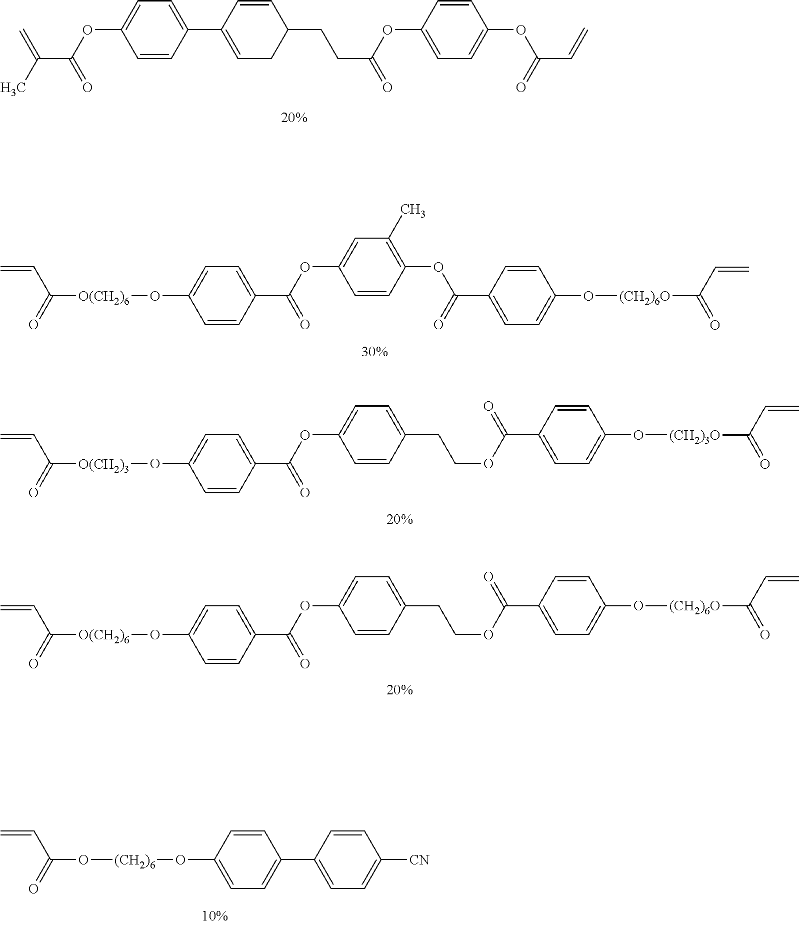

The polymerizable liquid crystal composition has preferable storage stability, and shows a nematic liquid cyrstalline phase. 3% of a photopolymerization initiator Irgacure 907 (manufactured by Ciba Speciality Chemicals Co., Ltd.) was added to the polymerizable liquid crystal composition to prepare a polymerizable liquid crystal composition (composition 5). An optically anisotropic material was obtained using the composition 5 according to the same method as Example 6. It was confirmed that in the optically anisotropic material which had gone through a rubbing process, the composition 5 was polymerized while maintaining a state where a molecule is uniformly aligned. A surface hardness (by JIS-S-K-5400) of the optically anisotropic material was HB. If a phase difference of the obtained optically anisotropic material before heating is 100%, when the optically anisotropic material was heated at a temperature of 240° C. for 1 hour, a phase difference was 85%, and a decreasing ratio of the phase difference was 15%.

As such, it is clear that a phase difference decreasing ratio of the optically anisotropic material that can be fabricated by the composition 5 of Comparative Example 2 was greater than that of the optically anisotropic material fabricated by the composition 2 of the present invention, and the heat resistance thereof was deteriorated. In addition, the surface hardness was HB, which is insufficient.

### Example 9

A liquid crystal composition LC-1 containing the compounds shown in the following was prepared. The compounds constituting the composition and ratios thereof to be contained are as follows.

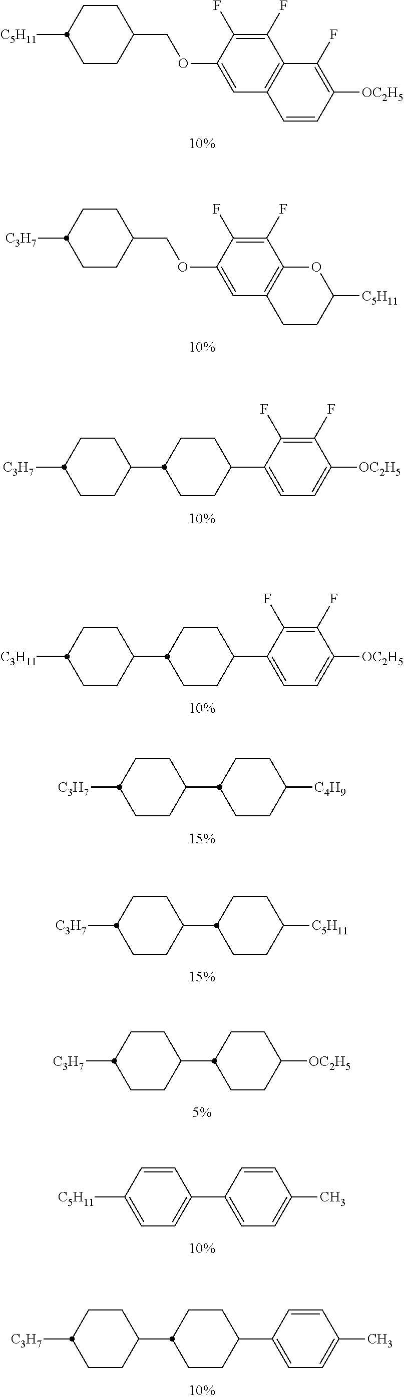

0.3% of the compound represented by the Formula (11) and synthesized in Example 3 was added to the liquid crystal composition LC-1. Precipitation did not occur even though the polymerizable liquid crystal composition was stored at a temperature of −10° C. for 1 week, and the storage stability was excellent. The composition was poured into a glass cell having polyimide and subjected to 3.5 μm of alignment processing, irradiated with 10 J of an ultraviolet ray, and then the liquid crystal composition was extracted from the glass cell. An analysis was performed on a residual monomer with high performance liquid chromatography, but the result was under the detection limit.

### Example 10

0.3% of the compound represented by the Formula (24) and synthesized in Example 7 was added to the liquid crystal composition LC-1. Precipitation did not occur even though the polymerizable liquid crystal composition was stored at a temperature of −10° C. for 1 week, and the storage stability was excellent. The composition was poured into a glass cell having polyimide and subjected to 3.5 μm of alignment process, irradiated with 5 J of an ultraviolet ray, and then the liquid crystal composition was extracted from the glass cell. An analysis was performed on a residual monomer with high performance liquid chromatography, but the result was under the detection limit.

### Comparative Example 3

0.3% of the compound represented by the Formula (25) was added to the liquid crystal composition LC-1. The composition was poured into a glass cell having polyimide and subjected to 3.5 μm of alignment process, irradiated with 10 J of an ultraviolet ray, and then the liquid crystal composition was extracted from the glass cell. An analysis was performed on a residual monomer with high performance liquid chromatography, but the result was under the detection limit. However, a precipitate of the liquid crystal composition was visually observed, when the composition was stored at a temperature of −10° C. for 3 days, and the solubility was poor.

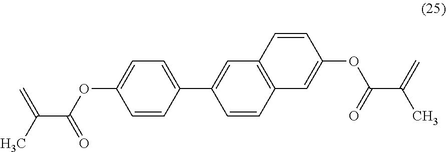 [SMILES: C=C(C)C(=O)Oc1ccc(-c2ccc3cc(OC(=O)C(=C)C)ccc3c2)cc1]

1. A polymerizable compound represented by General Formula (I): 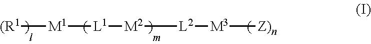
(in General Formula (I), Z represents a hydrogen atom, a C1 to C8 alkyl group, a C1 to C8 halogenated alkyl group, a C1 to C8 alkoxy group, a C1 to C8 halogenated alkoxy group, halogen, a cyano group, a nitro group, or —S¹—R², the S¹ is at least one linking group selected from the group consisting of a single bond and a C1 to C12 alkylene group, one —CH₂— or not-adjacent two or more —CH₂— in the alkylene group may be substituted with —O—, —COO—, —OCO—, or —OCOO—, R¹ and R² each independently represent a hydrogen atom or any one of the following Formulas (R-I) to (R-IX): 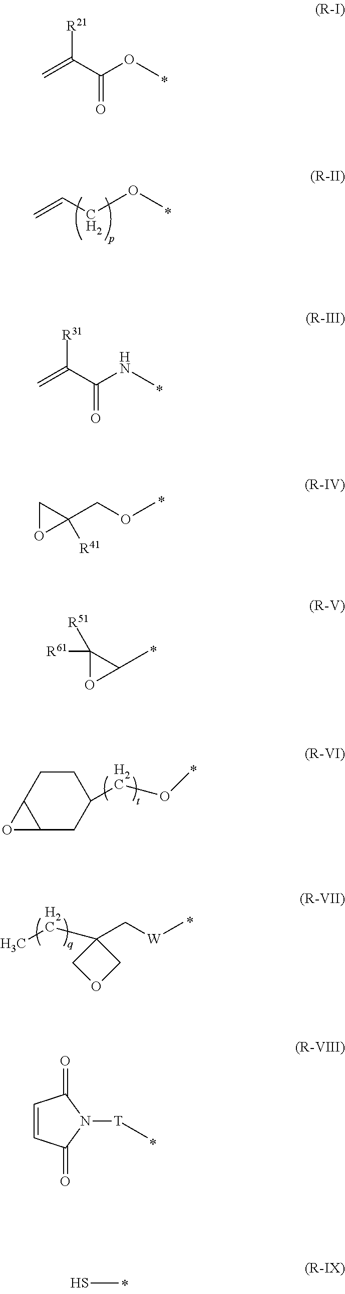
in the Formulas (R-I) to (R-IX), R²¹, R³¹, R⁴¹, R⁵¹ and R⁶¹ each independently represent a hydrogen atom, a C1 to C5 alkyl group, or a C1 to C5 halogenated alkyl group, W is a single bond, —O—, or a methylene group, T is a single bond, or —COO—, p, t, and q each independently are 0, 1, or 2,
L¹ and L² each independently represent a single bond, —O—, —S—, —CH₂—, —OCH₂—, —CH₂O—, —CO—, —C₂H₄—, —COO—, —OCO—, —OCOOCH₂—, —CH₂OCOO—, —OCH₂CH₂O—, —CO—NR^{a}—, —NR^{a}—CO—, —SCH₂—, —CH₂S—, —CH═CR^{a}—COO—, —CH═CR^{a}—OCO—, —COO—CR^{a} CH—, —OCO—CR^{a}═CH—, —COO—CR^{a}═CH—COO—, —COO—CR^{a}—CH—OCO—, —OCO—CR^{a}═CH—COO—, —OCO—CR^{a}═CH—OCO—, —COOC₂H₄—, —OCOC₂H₄—, —C₂H₄OCO—, —(CH₂)ⱼ—C(═O)—O—, —(CH₂)ⱼ—O—(C═O)—, —O—(C═O)—(CH₂)ⱼ—, —(C═O)—O—(CH₂)ⱼ—, —CH₂OCO—, —COOCH₂—, —OCOCH₂—, —CH—CH—, —CF═CF—, —CF═CH—, —CH═CF—, —CF₂—, —CF₂O—, —OCF₂—, —CF₂CH₂—, —CH₂CF₂—, —CF₂CF₂—, or —C≡C— (in the formulas, R^{a}s each independently represent a hydrogen atom or a C1 to C4 alkyl group, and in the formulas, j represents an integer of 1 to 4),
M¹ and M³ each independently represent an aromatic ring or an aliphatic ring, M² represents a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, or a 1,3-dioxane-2,5-diyl group, and the M¹, M², and M³ each independently may not be substituted or may be substituted with a C1 to C8 alkyl group, a C1 to C8 halogenated alkyl group, a C1 to C8 alkoxy group, halogen, a cyano group, or a nitro group,
l and n each independently represent an integer of 0 to 4, and satisfy 1+n≧1 (provided that, when l represents 0, R¹ is a hydrogen atom, and Z has any one group of Formulas (R-I) to (R-IX), and when n represents 0, R¹ has any one group of Formulas (R-I) to (RIX)), and
m represents an integer of 1 to 4, and when m is equal to or more than 2, L¹ and M², of which there are two each, may be the same as each other or different from each other, but at least one of L¹ represents a single bond.)

2. The polymerizable compound according to claim 1,
wherein in General Formula (I), L¹ represents —OCH₂—, —CH₂O—, —COO—, —OCO—, —C₂H₄—, —C≡C—, —OCF₂—, —CF₂O—, or a single bond, and
M¹ and M² each independently represent a 1,4-cyclohexylene group, a 1,4-phenylene group, or a naphthalene-2,6-diyl group, M³ represents a 1,3,5-benzenetriyl group, a 1,3,4-benzenetriyl group, a 1,3,4-cyclohexanetriyl group or a 1,3,5-cyclohexanetriyl group, M¹, M² and M³ each independently may be substituted with an alkyl group, a halogenated alkyl group, an alkoxy group, a halogenated alkoxy group, halogen, a cyano group, or a nitro group, and m represents 1 or 2.

3. The polymerizable compound according to claim 1,
wherein in General Formula (I), Z represents —S¹—R².

4. The polymerizable compound according to claim 3,
wherein in General Formula (I), R¹ is a Formula (R-I), and the R²¹ represents a C1 to C5 alkyl group, and
R² is a Formula (R-1), and the R²¹ represents a hydrogen atom.

5. The polymerizable compound according to claim 1,
wherein L² represents —COOC₂H₅— or —OCOC₂H₄—, M³ is a 1,3,5-benzenetriyl group, or a 1,3,4-benzenetriyl group, and m is 1.

6. A polymerizable composition comprising the polymerizable compound according to claim 1.

7. A liquid crystalline polymerizable composition comprising the polymerizable composition according to claim 6, which exhibits a liquid cyrstalline phase.

8. A liquid crystal composition containing the polymerizable compound according to claim 1, further comprising:
a non-polymerizable liquid crystal compound.

9. A liquid crystal composition containing the polymerizable compound according to claim 1 comprising:
a polymerizable compound used for a liquid crystal display element including a liquid crystal layer, a transparent electrode, and a polarizing plate formed between a pair of substrates,
wherein a liquid crystal alignment ability is imparted by polymerizing the polymerizable compound within the liquid crystal layer formed by filling a space formed between the pair of substrates with the liquid crystal composition containing the polymerizable compound.

10. An optically anisotropic material formed by polymerizing the a liquid crystal composition containing the polymerizable compound according to claim 7.

11. A liquid crystal display element to which liquid crystal alignment ability is imparted by using the liquid crystal composition containing the polymerizable compound according to claim 8, and polymerizing the polymerizable compound in the liquid crystal composition containing the polymerizable compound.
